SEQUENCE LISTING (59 sequences)

SEQ ID NO: 1 - protein, 100 aa, Artificial
  Dengue virus 1 Consensus Sequence Alignment of DEN1/dIII polyprotein
KGMSYVMCTGSFKLEKEVAETQHGTVLVQXKYEGTDAPCKIPFSTQDEKGXTQNGRLITANPIVTDKEKPVNIEAEPPFGESYIVXGAGEKALKLSWFKK

SEQ ID NO: 2 - protein, 100 aa, Artificial
  Dengue virus 1 DEN1/dIII polyprotein
KGMSYVMCTGSFKLEKEVAETQHGTVLVQVKYEGTDAPCKIPFSTQDEKGVTQNGRLITANPIVTDKEKPVNIEAEPPFGESYIVVGAGEKALKLSWFKK

SEQ ID NO: 3 - protein, 100 aa, Artificial
  Dengue virus 1 DEN1/dIII polyprotein
KGMSYVMCTGSFKLEKEVAETQHGTVLVQVKYEGTDAPCKIPFSTQDEKGITQNGRLITANPIVTDKEKPVNIEAEPPFGESYIVVGAGEKALKLSWFKK

SEQ ID NO: 4 - protein, 100 aa, Artificial
  Dengue virus 1 DEN1/dIII polyprotein
KGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKK

SEQ ID NO: 5 - protein, 100 aa, Artificial
  Dengue virus 1 DEN1/dIII polyprotein
KGMSYVMCTGSFKLEKEVAETQHGTVLVQIKYEGTDAPCKIPFSTQDEKGATQNGRLITANPIVTDKEKPVNIEAEPPFGESYIVIGAGEKALKLSWFKK

SEQ ID NO: 6 - protein, 100 aa, Artificial
  Dengue virus 1 DEN1/dIII polyprotein
KGMSYVMCTGSFKLEKEVAETQHGTVLVQIKYEGTDAPCKIPFSTQDEKGVTQNGRLITANPIVTDKEKPVNIEAEPPFGESYIVIGAGEKALKLSWFKK

SEQ ID NO: 7 - protein, 100 aa, Artificial
  Dengue virus 2 Consensus Sequence Alignment of DEN2/dIII polyprotein
KGMSYSMCTGKFKXVKEIAETQHGTIVXRVQYEGDGSPCKIPFEIXDLEKRHVLGRLITVNPIVTEKDSPXNIEAEPPFGDSYXIIGVEPGQLKLNWFKK

SEQ ID NO: 8 - protein, 100 aa, Artificial
  Dengue virus 2 of DEN2/dIII polyprotein
KGMSYSMCTGKFKVVKEIAETQHGTIVVRVQYEGDGSPCKIPFEIMDLEKRHVLGRLITVNPIVTEKDSPVNIEAEPPFGDSYIIIGVEPGQLKLNWFKK

SEQ ID NO: 9 - protein, 100 aa, Artificial
  Dengue virus 2 of DEN2/dIII polyprotein
KGMSYSMCTGKFKVVKEIAETQHGTIVVRVQYEGDGSPCKIPFEIMDLEKRHVLGRLITVNPIVTEKDSPVNIEAEPPFGDSYVIIGVEPGQLKLNWFKK

SEQ ID NO: 10 - protein, 100 aa, Artificial; identical to SEQ ID NO: 8
  Dengue virus 2 of DEN2/dIII polyprotein

SEQ ID NO: 11 - protein, 100 aa, Artificial
  Dengue virus 2 of DEN2/dIII polyprotein
KGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKK

SEQ ID NO: 12 - protein, 100 aa, Artificial
  Dengue virus 2 of DEN2/dIII polyprotein
KGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKK

SEQ ID NO: 13 - protein, 100 aa, Artificial
  Dengue virus 2 of DEN2/dIII polyprotein
KGMSYSMCTGKFKIVKEIAETQHGTIVIRVQYEGDGSPCKIPFEITDLEKRHVLGRLITVNPIVTEKDSPVNIEAEPPFGDSYIIIGVEPGQLKLNWFKK

SEQ ID NO: 14 - protein, 100 aa, Artificial
  Dengue virus 2 of DEN2/dIII polyprotein
KGMSYSMCTGKFKIVKEIAETQHGTIVIRVQYEGDGSPCKIPFEIMDLEKRHVLGRLITVNPIVTEKDSPINIEAEPPFGDSYIIIGVEPGQLKLNWFKK

SEQ ID NO: 15 - protein, 100 aa, Artificial
  Dengue virus 3 Consensus Sequence Alignment of DEN3/dIII polyprotein
XGMSYAMCLNTFVLKKEVSETQHGTXLIKVEYKGEDAPCKIPFSTEDGQGKAHNGRLITANPVVTKKEEPVNIEAEPPFGESNIVIGXGDKALKINWYXX

SEQ ID NO: 16 - protein, 100 aa, Artificial
  Dengue virus 3 of DEN3/dIII polyprotein
KGMSYAMCLNTFVLKKEVSETQHGTLLIKVEYKGEDAPCKIPFSTEDGQGKAHNGRLITANPVVTKKEEPVNIEAEPPFGESNIVIGIGDKALKINWYRK

SEQ ID NO: 17 - protein, 100 aa, Artificial
  Dengue virus 3 of DEN3/dIII polyprotein
KGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYKK

SEQ ID NO: 18 - protein, 100 aa, Artificial
  Dengue virus 3 of DEN3/dIII polyprotein
KGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYRK

SEQ ID NO: 19 - protein, 100 aa, Artificial
  Dengue virus 3 of DEN3/dIII polyprotein
RGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYRK

SEQ ID NO: 20 - protein, 100 aa, Artificial
  Dengue virus 3 of DEN3/dIII polyprotein
KGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYKR

SEQ ID NO: 21 - protein, 100 aa, Artificial
  Dengue virus 3 of DEN3/dIII polyprotein
KGMSYAMCLNTFVLKKEVSETQHGTILIKVEYKGEDAPCKIPFSTEDGQGKAHNGRLITANPVVTKKEEPVNIEAEPPFGESNIVIGVGDKALKINWYKK

SEQ ID NO: 22 - protein, 100 aa, Artificial
  Dengue virus 4 Consensus Sequence Alignment of DEN4/dIII polyprotein
KGMSYTMCXGKFSIDKEMAETQHGTTVVKVKYEGAGAPCKXPIEIRDVNKEXVVGRXISSTPLAENTNSXTNIELEPPFGDSYIVIGVGNSALTLHWFRK

SEQ ID NO: 23 - protein, 100 aa, Artificial
  Dengue virus 4 of DEN4/dIII polyprotein
KGMSYTMCPGKFSIDKEMAETQHGTTVVKVKYEGAGAPCKVPIEIRDVNKEKVVGRIISSTPLAENTNSATNIELEPPFGDSYIVIGVGNSALTLHWFRK

SEQ ID NO: 24 - protein, 100 aa, Artificial
  Dengue virus 4 of DEN4/dIII polyprotein
KGMSYTMCSGKFSIDKEMAETQHGTTVVKVKYEGAGAPCKVPIEIRDVNKEKVVGRIISSTPLAENTNSATNIELEPPFGDSYIVIGVGNSALTLHWFRK

SEQ ID NO: 25 - protein, 100 aa, Artificial
  Dengue virus 4 of DEN4/dIII polyprotein
KGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRK

SEQ ID NO: 26 - protein, 100 aa, Artificial
  Dengue virus 4 of DEN4/dIII polyprotein
KGMSYTMCSGKFSIDKEMAETQHGTTVVKVKYEGAGAPCKIPIEIRDVNKEKVVGRIISSTPLAENTNSVTNIELEPPFGDSYIVIGVGNSALTLHWFRK

SEQ ID NO: 27 - protein, 100 aa, Artificial
  Dengue virus 4 of DEN4/dIII polyprotein
KGMSYTMCSGKFSIDKEMAETQHGTTVVKVKYEGAGAPCKVPIEIRDVNKEKVVGRVISSTPLAENTNSVTNIELEPPFGDSYIVIGVGNSALTLHWFRK

SEQ ID NO: 28 - protein, 100 aa, Artificial
  Dengue virus 4 of DEN4/dIII polyprotein
KGMSYTMCSGKFSIDKEMAETQHGTTVVKVKYEGAGAPCKVPIEIRDVNKEKVVGRVISSTPLAENTNSATNIELEPPFGDSYIVIGVGNSALTLHWFRK

SEQ ID NO: 29 - protein, 100 aa, Artificial
  Dengue virus 4 of DEN4/dIII polyprotein
KGMSYTMCSGKFSIDKEMAETQHGTTVVKVKYEGAGAPCKVPIEIRDVNKEXVVGRVISSTPLAENTNSATNIELEPPFGDSYIVIGVGNSALTLHWFRK

SEQ ID NO: 30 - protein, 100 aa, Artificial
  Yellow fever virus, Consensus sequence of YFV/dIII polyprotein
KGTSYKXCTDKMXFVKNPTDTXHGTXVMQVKVXKGAPCXIPVXVADDLTAXXNKGILVTVNXIASTNXDEVLIEVNPPFGDSYIIXGXGDSRLTYQWHKE

SEQ ID NO: 31 - protein, 100 aa, Artificial
  Yellow fever virus of YFV/dIII polyprotein
KGTSYKMCTDKMSFVKNPTDTGHGTVVMQVKVPKGAPCKIPVIVADDLTAAINKGILVTVNPIASTNDDEVLIEVNPPFGDSYIIIGTGDSRLTYQWHKE

SEQ ID NO: 32 - protein, 100 aa, Artificial
  Yellow fever virus of YFV/dIII polyprotein
KGTSYKMCTDKMSFVKNPTDTDHGTVVMQVKVPKGAPCKIPVIVADDLTAAINKGILVTVNPIASTNDDEVLIEVNPPFGDSYIIVGTGDSRLTYQWHKE

SEQ ID NO: 33 - protein, 100 aa, Artificial
  Yellow fever virus of YFV/dIII polyprotein
KGTSYKMCTDKMSFVKNPTDTGHGTVVMQVKVPKGAPCKIPVIVADDLTAAINKGILVTVNPIASTNDDEVLIEVNPPFGDSYIIVGTGDSRLTYQWHKE

SEQ ID NO: 34 - protein, 100 aa, Artificial
  Yellow fever virus of YFV/dIII polyprotein
KGTSYKMCTDKMSFVKNPTDTGHGTAVMQVKVPKGAPCRIPVMVADDLTASVNKGILVTVNPIASTNEDEVLIEVNPPFGDSYIIVGTGDSRLTYQWHKE

SEQ ID NO: 35 - protein, 100 aa, Artificial
  Yellow fever virus of YFV/dIII polyprotein
KGTSYKMCTDKMSFVKNPTDTGHGTAVMQVKVPKGAPCGIPVMVADDLTASVNKGILVTVNPIASTNEDEVLIEVNPPFGDSYIIVGTGDSRLTYQWHKE

SEQ ID NO: 36 - protein, 100 aa, Artificial
  Yellow fever virus of YFV/dIII polyprotein
KGTSYKMCTDKMSFVKNPTDTGHGTAVMQVKVPKGAPCRIPVMVADDLTAAVNKGILVTVNPIASTNDDEVLIEVNPPFGDSYIIVGTGDSRLTYQWHKE

SEQ ID NO: 37 - protein, 100 aa, Artificial
  Yellow fever virus of YFV/dIII polyprotein
KGTSYKMCTDKMSFVKNPTDTGHGTVVMQVKVPKGAPCKIPVIVADDLTAAVNKGILVTVNPIASTNDDEVLIEVNPPFGDSYIIVGTGDSRLTYQWHKE

SEQ ID NO: 38 - protein, 100 aa, Artificial
  Yellow fever virus of YFV/dIII polyprotein
KGTSYKMCTDKMSFVKNPTDTGHGTVVMQVKVPKGAPCRIPVIVADDLTAAINKGILVTVNPIASTNDDEVLIEVNPPFGDSYIIVGTGDSRLTYQWHKE

SEQ ID NO: 39 - protein, 100 aa, Artificial
  Yellow fever virus of YFV/dIII polyprotein
KGTSYKICTDKMFFVKNPTDTGHGTVVMQVKVSKGAPCRIPVIVADDLTAAINKGILVTVNPIASTNDDEVLIEVNPPFGDSYIIVGRGDSRLTYQWHKE

SEQ ID NO: 40 - protein, 99 aa, Artificial
  Yellow fever virus of YFV/dIII polyprotein
SYKICTDKMFFVKNPTDTGHGTVVMQVKVSKGAPCRIPVIVADDLTAAINKGILVTVNSIASTNDDEVLIEVNPPFGDSYIIVGRGDSRLTYQWHKEGS

SEQ ID NO: 41 - protein, 100 aa, Artificial; identical to SEQ ID NO: 39
  Yellow fever virus of YFV/dIII polyprotein

SEQ ID NO: 42 - protein, 102 aa, Artificial
  West nile virus, Consensus sequence of WNV/dIII polyprotein
KGTTYGVCSKAFKFXXTPADTGHGTVVLELQYTGXDGPCKVPISSVASLNDLTPVGRLVTVNPFVSVATANXKVLIELEPPFGDSYIVVGRGEQQINHHWHK

SEQ ID NO: 43 - protein, 102 aa, Artificial
  West nile virus of WNV/dIII polyprotein
KGTTYGVCSKAFKFARTPADTGHGTVVLELQYTGTDGPCKVPISSVASLNDLTPVGRLVTVNPFVSVATANSKVLIELEPPFGDSYIVVGRGEQQINHHWHK

SEQ ID NO: 44 - protein, 102 aa, Artificial
  West nile virus of WNV/dIII polyprotein
KGTTYGVCSKAFKFARTPADTGHGTVVLELQYTGKDGPCKVPISSVASLNDLTPVGRLVTVNPFVSVATANSKVLIELEPPFGDSYIVVGRGEQQINHHWHK

SEQ ID NO: 45 - protein, 102 aa, Artificial
  West nile virus of WNV/dIII polyprotein
KGTTYGVCSKAFKFAGTPADTGHGTVVLELQYTGTDGPCKVPISSVASLNDLTPVGRLVTVNPFVSVATANSKVLIELEPPFGDSYIVVGRGEQQINHHWHK

SEQ ID NO: 46 - protein, 102 aa, Artificial
  West nile virus of WNV/dIII polyprotein
KGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHK

SEQ ID NO: 47 - protein, 102 aa, Artificial; identical to SEQ ID NO: 46
  West nile virus of WNV/dIII polyprotein

SEQ ID NO: 48 - protein, 102 aa, Artificial
  Japanese Encephalitis Virus, Consensus sequence of JEV/dIII polyprotein
KGTTYGMCTXKFSFAKNPADTGHGTVVIELSYXGXDGPCKIPIVSXASLNDXTPXGRLVTVNPFVATSSANSKVLVEMEPPFGDSYIVVGRXXKQINHHWHK

SEQ ID NO: 49 - protein, 102 aa, Artificial
  Japanese Encephalitis Virus of JEV/dIII polyprotein
KGTTYGMCTKKFSFAKNPADTGHGTVVIELSYSGSDGPCKIPIVSVASLNDMTPVGRLVTVNPFVATSSANSKVLVEMEPPFGDSYIVVGRGDKQINHHWHK

SEQ ID NO: 50 - protein, 102 aa, Artificial
  Japanese Encephalitis Virus of JEV/dIII polyprotein
KGTTYGMCTEKFSFAKNPADTGHGTVVIELSYSGSDGPCKIPIVSVASLNDMTPVGRLVTVNPFVATSSANSKVLVEMEPPFGDSYIVVGRGDKQINHHWHK

SEQ ID NO: 51 - protein, 102 aa, Artificial
  Japanese Encephalitis Virus of JEV/dIII polyprotein
KGTTYGMCTGKFSFAKNPADTGHGTVVIELSYCGSDGPCKIPIVSVASLNDMTPVGRLVTVNPFVATSSANSKVLVEMEPPFGDSYIVVGRGDKQINHHWHK

SEQ ID NO: 52 - protein, 102 aa, Artificial
  Japanese Encephalitis Virus of JEV/dIII polyprotein
KGTTYGMCTGKFSFAKNPADTGHGTVVIELSYSGSDGPCKIPIVSAASLNDMTPVGRLVTVNPFVATSSANSKVLVEMEPPFGDSYIVVGRGDKQINHHWHK

SEQ ID NO: 53 - protein, 102 aa, Artificial
  Japanese Encephalitis Virus of JEV/dIII polyprotein
KGTTYGMCTGKFSFAKNPADTGHGTVVIELSYSGRDGPCKIPIVSVASLNDMTPVGRLVTVNPFVATSSANSKVLVEMEPPFGDSYIVVGRGDKQINHHWHK

SEQ ID NO: 54 - protein, 102 aa, Artificial; identical to SEQ ID NO: 53
  Japanese Encephalitis Virus of JEV/dIII polyprotein

SEQ ID NO: 55 - protein, 102 aa, Artificial
  Japanese Encephalitis Virus of JEV/dIII polyprotein
KGTTYGMCTGKFSFAKNPADTGHGTVVIELSYSGSDGPCKIPIVSVASLNDLTPVGRLVTVNPFVATSSANSKVLVEMEPPFGDSYIVVGRGDKQINHHWHK

SEQ ID NO: 56 - protein, 102 aa, Artificial
  Japanese Encephalitis Virus of JEV/dIII polyprotein
KGTTYGMCTGKFSFAKNPADTGHGTVVIELSYSGSDGPCKIPIVSVASLNDLTPVGRLVTVNPFVATSSANSKVLVEMEPPFGDSYIVVGRGNKQINHHWHK

SEQ ID NO: 57 - protein, 102 aa, Artificial
  Japanese Encephalitis Virus of JEV/dIII polyprotein
KGTTYGMCTGKFSFAKNPADTGHGTVVIELSYSGSDGPCKIPIVSVASLNDMTPVGRLVTVNPFVATSSANSKVLVEMEPPFGDSYIVVGRGDKQINHHWHK

SEQ ID NO: 58 - protein, 102 aa, Artificial
  Japanese Encephalitis Virus of JEV/dIII polyprotein
KGTTYGMCTGKFSFAKNPADTGHGTVVIELSYSGSDGPCKIPIVSVASLNDMTPAGRLVTVNPFVATSSANSKVLVEMEPPFGDSYIVVGREDKQINHHWHK

SEQ ID NO: 59 - DNA, 297 bp, Artificial
  Yellow Fever Virus 17D dIII nucleotide sequence
tcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaactccatcgcctcaaccaatgatgatgaagtgctgattgaggtgaacccaccttttggagacagctacattatcgttgggagaggagattcacgtctcacttaccagtggcacaaagagggatcc

(19) United States
(12) Patent Application Publication
(10) Pub. No.: US 2013/0295162 A1
(43) Pub. Date: Nov. 7, 2013

(54) FLAVIVIRUS DOMAIN III VACCINE

(71) Applicants: Jacob J. Schlesinger, Pittsford, NY (US); Xia Jin, Fairport, NY (US); Robert C. Rose, Cleveland, OH (US); Olivia K.T. Block, Rochester, NY (US); W.W. Shanaka I. Rodrigo, Colombo (LK)

(72) Inventors: Jacob J. Schlesinger, Pittsford, NY (US); Xia Jin, Fairport, NY (US); Robert C. Rose, Cleveland, OH (US); Olivia K.T. Block, Rochester, NY (US); W.W. Shanaka I. Rodrigo, Colombo (LK)

(73) Assignee: UNIVERSITY OF ROCHESTER, Rochester, NY (US)

(21) Appl. No.: 13/876,893

(22) PCT Filed: Oct. 3, 2011
(86) PCT No.: PCT/US11/54531

§ 371 Date: Jul. 26, 2013

Related U.S. Application Data
(60) Provisional application No. 61/388,780, filed on Oct. 1, 2010.

(51) Int. Cl. A61K 39/12

(52) U.S. Cl. CPC A61K 39/12
USPC 424/450; 424/186.1; 435/69.3

(57) ABSTRACT

The disclosure provides a tetravalent Dengue virus vaccine, and methods of inducing a immune response against a Flavivirus such as Dengue virus 1-4 using the vaccine. The disclosure also provides methods of making a vaccine by introducing into a host cell a transgene that encodes a protein comprising Dengue domain III polypeptides, or, by purified dill polypeptides specific for Dengue serotypes 1-4, Yellow Fever Virus, West Nile Virus, Japanese Encephalitis Virus, and Flaviviruses.

This application claims the benefit of U.S. Provisional Patent Application Ser. No. 61/388,780, filed Oct. 1, 2010, which is hereby incorporated by reference in its entirety.

### FIELD OF THE INVENTION

The present invention relates generally to vaccine formulations, and more specifically to a Flavivirus vaccine, its use, and methods of manufacture.

### BACKGROUND OF THE INVENTION

Among medically important flaviviviruses the mosquito-borne dengue viruses (“DENVs”) are notable for their wide global distribution and unusually high incidence of human infection that may be complicated by dengue hemorrhagic fever/shock syndrome (“DHF/DSS”) (see Halstead, S B., “Neutralization and Antibody-Dependent Enhancement of Dengue Viruses,” Adv. Virus Res. 60:421-67 (2003)). DENVs exist as four serologically distinct, but antigenically related single positive-strand RNA viruses (DENV1-4). The four serotypes of dengue virus—dengue virus type 1 (DEN1), DEN2, DEN3, and DEN4—annually cause an estimated 50 to 100 million cases of dengue fever and 500,000 cases of the more severe form of dengue virus infection known as DHF/DSS (Gubler et al., “Impact of Dengue/Dengue Haemorrhagic Fever on the Developing World,” Adv. Virus. Res. 53:35-70 (1999)). Dengue virus is widely distributed throughout the tropical and subtropical regions of the world, and the number of dengue virus infections continues to increase due to the expanding range of its Aedes aegypli mosquito vector. Although a number of vaccines are currently undergoing trials, a commercial vaccine is not yet available for the control of dengue disease despite its importance as a reemerging disease. The goal of immunization is to protect against dengue virus disease by the induction of a long-lived neutralizing antibody response against each of the four serotypes. Simultaneous protection against all four serotypes is required, since an increase in disease severity can occur in persons with preexisting antibodies to a heterotypic dengue virus.

Currently, there is no licensed vaccine or proven antiviral treatment. Cross-reactive antibodies that confer relatively brief heterologous DENV protection are generated early in the course of primary DENV infection (Sabin, A., “Research on Dengue During World War II” Am. Trop. Med. Hyg. 1:30-50 (1952)), whereas, durable protective immunity is conferred by potent anti-virion neutralizing antibodies that are DENV serotype-specific. Thus, sequential infection with different DENV serotypes is common in endemic regions where multiple DENV serotypes co-circulate. Paradoxically, antibodies against DENV may predispose to DHF/DSS during a second DENV infection, an outcome hypothesized to involve so-called antibody-dependent enhancement (“ADE”) (Halstead, S B., “Neutralization and Antibody-Dependent Enhancement of Dengue Viruses,” Adv. Virus Res. 60:421-67 (2003)). In this situation, amplified viral replication occurs when infectious DENV immune complexes comprised of weakly or non-neutralizing cross-reactive antibodies enter and replicate in monocytic target cells after Fcγ receptor (FcγR) engagement.

The Dengue virus genome contains a single open reading frame encoding a polyprotein which is processed by proteases of both viral and cellular origin into three structural proteins (C, prM, and E) and at least seven nonstructural (NS) proteins. Neutralizing antibodies are largely directed against the DENV virion envelope E protein which is comprised of the three structurally distinct domains (dI, dII, dIII) that subserve host cell attachment (E dIII) or post-entry endosomal fusion (E dI/II) (see Pierson et al., “Structural Insights Into The Mechanisms of Antibody-Mediated Neutralization of Flavivirus Infection Implications For Vaccine Development,” Cell Host Microbe 4(3):229-38 (2008)). A precursor membrane protein (prM) associates with E dI/II on immature virions, protecting them against intracellular fusion in the course of their assembly and release from the host cell (Kuhn et al., “Structure of Dengue Virus: Implications for Flavivirus Organization, Maturation, and Fusion,” Cell 108 (5):717-25 (2002)). Importantly, in human infection DENV prM appears to generate predominantly DENV cross-reactive antibodies that exhibit little or no neutralizing activity and strongly promote ADE by rendering antibody-complexed immature virions infectious (Dejnirattisai et al., “Cross-Reacting Antibodies Enhance Dengue Virus Infection in Hiumans,” Science 328(5979):745-8 (2010); Rodenhuis-Zybert et al., “Immature Dengue Virus: A Veiled Pathogen?,” PLoS. Pathog. 6(1):e1000718 (2010)).

More broadly flavivirus cross-reactive determinants that subserve both neutralization (typically weak) and ADE are concentrated on E dI/II. Conversely, DENV dIII incorporates mainly serotype specific determinants. These include dIII lateral ridge epitopes that are recognized by a number of especially potent DENV serotype specific neutralizing mouse monoclonal antibodies (mAbs) (Sukupolvi-Petty et al., “Type- and Subcomplex-Specific Neutralizing Antibodies Against Domain III of Dengue Virus Type 2 Envelope Protein Recognize Adjacent Epitopes,” J. Virol. 81(23):12816-26 (2007)) including those with therapeutic potential (Shrestha et al., “The Development of Therapeutic Antibodies That Neutralize Homologous and Heterologous Genotypes of Dengue Virus Type 1,” PLoS. Pathog. 6(4):e1000823 (2010)).

To date, however, no tetravalent Dengue vaccine has been generated that is capable of inducing a balanced, neutralizing immune response characterized by a PRNT₅₀ of at least about 150 for each of the Dengue serotypes. The present invention is directed to overcoming these and other deficiencies in the art.

### SUMMARY OF THE INVENTION

A first aspect of the present invention relates to a tetravalent Dengue virus vaccine that includes a Dengue domain III (dIII) polypeptide for each of DEN1 to DEN4, where the vaccine induces a neutralizing antibody response against each of DEN1 to DEN4 that exceeds a PRNT₅₀ value of 150. As used herein, a particular PRNT₅₀ value refers to the 50% plaque reduction neutralizing titer, which is the geometrical reciprocal of the serum dilution yielding 50% reduction in plaque number as measured according to a plaque assay.

A second aspect of the present invention relates to a method of inducing a neutralizing immune response against Dengue virus strains 1-4 in a subject that includes administering to the subject a tetravalent Dengue virus vaccine according to a first aspect of the invention in an amount effective to induce a neutralizing immune response against each of DEN1 to DEN4 that exceeds a PRNT₅₀ value of 150.

A third aspect of the present invention relates to a method of making a tetravalent Dengue virus vaccine according to the first aspect of the invention. This method includes combining, with a pharmaceutically acceptable vehicle, purified dIII polypeptide specific for Dengue serotypes 1-4 in effective amounts to induce a neutralizing immune response against each of DEN1 to DEN4 that exceeds PRNT₅₀ of 150.

A fourth aspect of the present invention relates to a monovalent or multivalent Flavivirus vaccine that includes a Flavivirus E protein domain III polypeptide for one or more than one serotype of the Flavivirus, wherein the vaccine induces a neutralizing antibody response against each of the one or more than one serotype of Flavivirus that exceeds PRNT₅₀ value of 150.

A fifth aspect of the present invention relates to a method of inducing a neutralizing immune response against Flavivirus in a subject that includes administering to the subject a Flavivirus vaccine according to the fourth aspect of the invention in an amount effective to induce a neutralizing immune response against each of the one or more than one serotype of Flavivirus that exceeds PRNT₅₀ of 150.

A sixth aspect of the present invention relates to a method of making a Flavivirus vaccine according to the fourth aspect of the invention. This method includes combining, with a pharmaceutically acceptable vehicle, purified dIII polypeptide specific for one or more than one serotype of the Flavivirus in effective amounts to induce a neutralizing immune response against each of the one or more than one serotype of the Flavivirus that exceeds PRNT₅₀ of 150

A seventh aspect of the invention relates to a multivalent vaccine that includes an effective amount of a Dengue virus domain III polypeptide for each of DEN1 to DEN4, an effective amount of a Yellow Fever virus domain III polypeptide, and a pharmaceutically acceptable carrier. In certain embodiments, the multivalent vaccine induces a neutralizing antibody response against each of DEN1 to DEN4 and YFV that exceeds a PRNT₅₀ value of 150.

Dengue viruses co-circulate as four serologically distinct viruses (DENV1-4) that commonly infect individuals sequentially. Current DENV candidate vaccines incorporate the entire virion envelope E protein (E) ectodomain thereby stimulating both DENV serotype-specific and cross-reactive antibodies. Because the latter may enhance naturally acquired infection, such vaccine formulations must be tetravalent. The Examples presented herein demonstrate the efficacy of a tetravalent dIII polypeptide vaccine that achieves a neutralizing immune response that is substantially improved over other Dengue subunit vaccines, including prior dIII subunit vaccines. The Examples demonstrate the neutralizing and enhancing antibody response to dIII polypeptides, in which serotype-specific neutralizing determinants are concentrated. High-yield insect cell expression of the dIII polypeptides to each DENV serotype were obtained and characterized. Mice immunized with these recombinant DENV-dIII polypeptides individually, and in tetravalent combination, produce serotype-specific IgG1 neutralizing antibodies. While the immune response exhibits measurable DENV enhancing activity in FcγR-bearing cells, and also mediated measurable levels of ADE in FcγR-positive cells, the ADE-response is significantly diminished relative to prior Dengue vaccine formulations. Based on the success against Dengue, the present invention contemplates use of this same strategy against other Flaviviruses, including without limitation West Nile virus, Japanese Encephalitis virus, Kunjin virus, Murray Valley Encephalitis virus, Uganda-S virus, Yellow Fever virus, Tick-borne Encephalitis virus, Hepatitis C virus, and Louping-ill virus.

### BRIEF DESCRIPTION OF THE DRAWINGS

FIG. 1 is a ClustalW multiple sequence alignment of domain III polypeptides of DENV1 isolates, which was prepared using default settings. A consensus sequence (SEQ ID NO: 1) was introduced to the ClustalW-generated alignment subsequent to performing the alignment. The domain III sequences of DENV1 isolates were obtained from Genbank Accessions ACF49259 (SEQ ID NO: 2), ABR13878 (SEQ ID NO: 3), AF180817 (SEQ ID NO: 4), ACY70792 (SEQ ID NO: 5), and ACW82925 (SEQ ID NO: 6). Each of the above-identified Genbank Accession Nos. is hereby incorporated by reference in its entirety.

FIG. 2 is a ClustalW multiple sequence alignment of domain III polypeptides of DENV2 isolates, which was prepared using default settings. A consensus sequence (SEQ ID NO: 7) was introduced to the ClustalW-generated alignment subsequent to performing the alignment. The domain III sequences of DENV2 isolates were obtained from Genbank Accessions AAA17500 (SEQ ID NO: 8), ABQ18242 (SEQ ID NO: 9), AAA17509 (SEQ ID NO: 10), NC_{—}001474 (SEQ ID NO: 11), ADK37501 (SEQ ID NO: 12), AAT35547 (SEQ ID NO: 13), and AAS49675 (SEQ ID NO: 14). Each of the above-identified Genbank Accession Nos. is hereby incorporated by reference in its entirety.

FIG. 3 is a ClustalW multiple sequence alignment of domain III polypeptides of DENV3 isolates, which was prepared using default settings. A consensus sequence (SEQ ID NO: 15) was introduced to the ClustalW-generated alignment subsequent to performing the alignment. The domain III sequences of DENV3 isolates were obtained from Genbank Accessions CAD91364 (SEQ ID NO: 16), AAC63314 (SEQ ID NO: 17), M93130 (SEQ ID NO: 18), ADK79072 (SEQ ID NO: 19), ABA25808 (SEQ ID NO: 20), and ABA25785 (SEQ ID NO: 21). Each of the above-identified Genbank Accession Nos. is hereby incorporated by reference in its entirety.

FIG. 4 is a ClustalW multiple sequence alignment of domain III polypeptides of DENV4 isolates, which was prepared using default settings. A consensus sequence (SEQ ID NO: 22) was introduced to the ClustalW-generated alignment subsequent to performing the alignment. The domain III sequences of DENV4 isolates were obtained from Genbank Accessions U18429 (SEQ ID NO: 23), ACY01658 (SEQ ID NO: 24), ACW83008 (SEQ ID NO: 25), ACY01661 (SEQ ID NO: 26), ACH61714 (SEQ ID NO: 27), AAN38651 (SEQ ID NO: 28), and AAN38652 (SEQ ID NO: 29). Each of the above-identified Genbank Accession Nos. is hereby incorporated by reference in its entirety.

FIG. 5 is a ClustalW multiple sequence alignment of domain III polypeptides of YFV isolates, which was prepared using default settings. A consensus sequence (SEQ ID NO: 30) was introduced to the ClustalW-generated alignment subsequent to performing the alignment. The domain III sequences of YFV isolates were obtained from Genbank Accessions AAC72235 (SEQ ID NO: 31), AAA99812 (SEQ ID NO: 32), AAT12476 (SEQ ID NO: 33), AAD45531 (SEQ ID NO: 34), AAD45534 (SEQ ID NO: 35), ADK47994 (SEQ ID NO: 36), AAA92704 (SEQ ID NO: 37), AAA99712 (SEQ ID NO: 38), and ACN41908 (SEQ ID NO: 39), Strain16562 (SEQ ID NO: 40), and Genbank Accession AAC35902 (SEQ ID NO: 41). Each of the above-identified Genbank Accession Nos. is hereby incorporated by reference in its entirety.

FIG. 6 is a ClustalW multiple sequence alignment of domain III polypeptides of WNV isolates, which was prepared using default settings. A consensus sequence (SEQ ID NO: 42) was introduced to the ClustalW-generated alignment subsequent to performing the alignment. The domain III sequences of WNV isolates were obtained from Genbank Accessions AAA48498 (SEQ ID NO: 43), AAT95390 (SEQ ID NO: 44), ABR19636 (SEQ ID NO: 45), ADL27943 (SEQ ID NO: 46), and ADL27940 (SEQ ID NO: 47). Each of the above-identified Genbank Accession Nos. is hereby incorporated by reference in its entirety.

FIG. 7 is a ClustalW multiple sequence alignment of domain III polypeptides of JEV isolates, which was prepared using default settings. A consensus sequence (SEQ ID NO: 48) was introduced to the ClustalW-generated alignment subsequent to performing the alignment. The domain III sequences of WNV isolates were obtained from Genbank Accessions AAQ73507 (SEQ ID NO: 49), AAQ73509 (SEQ ID NO: 50), AAP14894 (SEQ ID NO: 51), ACU42249 (SEQ ID NO: 52), AAF34187 (SEQ ID NO: 53), AAB51519 (SEQ ID NO: 54), AAQ73512 (SEQ ID NO: 55), AAQ73513 (SEQ ID NO: 56), BAF02840 (SEQ ID NO: 57), and AAA67164 (SEQ ID NO: 58). Each of the above-identified Genbank Accession Nos. is hereby incorporated by reference in its entirety.

FIGS. 8A-C show sequence homologies of DENV dIII proteins of strains 16007 (SEQ ID NO: 4), 16681 (SEQ ID NO: 11), 16562 (SEQ ID NO: 40), and 1036 (SEQ ID NO: 23). In FIG. 8A, details for the viruses used for the production of recombinant DENV dIII proteins are identified. In FIG. 8B, DENV dIII amino acid sequence alignments are shown (performed using ClustalW2 Software on default settings). Conserved amino acid residues are indicated in bold-faced type. FIG. 8C shows percent homology among DNV dIII sequences.

FIGS. 9A-C illustrate expression, purification, and characterization of DENV dIII proteins. FIG. 9A shows a representative Western Blot of cobalt metal affinity-purified DENV dIII protein (DENV2 dIII shown); P, cell pellet; SN, cell supernatant: E1, elution fraction 1; E2, elution fraction 2 (visualized using anti-6-HIS mAb). FIG. 9B shows SDS-PAGE analysis of purified DENV dIII proteins (DENV serotypes 1-4, stained with Coomassie Blue). FIG. 9C illustrates results of a Western Blot analysis of purified DENV dIII proteins by: serotype-specific monoclonal antibodies against DENV1 (DV1-E50); DENV2 (1F1): and DENV3 (8A1) or monospecific DENV1, DENV2, and DENV4 immune mouse sera (MIAF). Pooled sera from patients infected with multiple DENV serotypes (PHS) reacted with dIII of all DENV serotypes.

FIGS. 10A-D show antibody response to DENV2-dIII immunization in mice. FIG. 10A illustrates the mouse immunization schedule. FIGS. 10B-C illustrate ELISA endpoint titers of mouse sera collected on days −2, 12, 26, and 42 against: DENV2-dIII protein (FIG. 10B) or intact DENV2 virions (FIG. 10C). FIG. 10D shows neutralization endpoint titers of pooled DENV2 monovalent sera (n=5) tested against each DENV serotype by 50% plaque reduction neutralization (PRNT₅₀) assay on Vero cells. PRNT₅₀ titers were calculated by probit analysis. Geometric mean titers (GMT) are indicated (n=5).

FIGS. 11A-C illustrate antibody response in mice immunized with a tetravalent vaccine comprised of equal amounts of DENV dIII serotype specific proteins. ELISA endpoint titers of pre- and post-vaccination mouse sera determined against DENV1-4 dIII proteins (shown in FIG. 11A) or DENV2 virions (shown in FIG. 11B). FIG. 11C shows neutralization by pooled mouse immune sera (post-primary vaccination day 42) determined by 50% plaque reduction neutralization test (PRNT₅₀) assay in Vero cells. PRNT₅₀ titers were calculated by probit analysis. Geometric mean titers (GMT) are indicated (n=5).

FIGS. 12A-C show antibody responses to mixed-dose monovalent and tetravalent DENV dIII immunization in mice. 25 μg DENV1 dIII, 5 μg DENV2 dIII, 25 μg DENV3 dIII, and 50 μg DENV4 dIII doses were inoculated individually (monovalent) or in tetravalent mixture using a prime and double-boost schedule with sera collected on post-primary vaccination day 42. FIG. 12A shows pooled sera that correspond to each formulation and an anti-6-HIS mAb were tested for reactivity to each DENV dIII protein or to an irrelevant 6HIS-tagged protein, bacteriophage gpD (6HIS-gpD). Neutralization by immune serum from individual mice immunized by monovalent (shown in FIG. 12B) or tetravalent (shown in FIG. 12C) vaccination was determined by 50% plaque reduction neutralization test (PRNT₅₀) assay in Vero cells. PRNT₅₀ titers were calculated by probit analysis. Geometric mean titers (GMT) are indicated (n=5). **P=<0.05 (Kruskal-Wallis test).

FIGS. 13A-B show DENV2 specific IgG subclass distribution in mouse immune sera. The IgG subclass distribution in sera from mice immunized with tetravalent DENV dIII protein or live DENV2 virion was determined by ELISA using DENV2 dIII protein (shown in FIG. 13A) or DENV2 virions (shown in FIG. 13B) in the solid phase.

FIGS. 14A-E illustrate that antibody-dependent enhancement is mediated by DENV dIII mouse immune serum in FcγR-expressing cell lines. In FIGS. 14A and 14B, K562 cells or U937 cells were infected with DENV2 in the presence or absence of serial 10-fold dilutions of sera from mice immunized with tetravalent DENV dIII vaccine. FIGS. 14C and 14D show relative peak ADE levels among monotypic DENV dIII immune sera; single serum dilutions used correspond to peak enhancement titers obtained from preliminary ranging experiments with both cell types. Non-immune serum collected before vaccination served as a control. FIG. 14E shows neutralization and ADE by IgG2a mAb 1F1 in K562 or U937 cells. After 2 days in stationary culture, anti-E mAb (7E1) stained cells were counted by a BD LSRII instrument and analyzed using FlowJo software. Fold differences in percentages of anti-E antibody stained cells for each condition are indicated from experiments performed in triplicate. Dotted lines indicate infection in the absence of mouse serum. Error bars display SD of triplicate determinations (invisible for U937 determinations because of low variation). Results are representative of at least two experiments performed with each cell type.

FIGS. 15A-B illustrate antibody response to YF17D dIII immunization. FIG. 15A illustrates the mouse immunization schedule. FIG. 15B illustrates neutralization endpoint titers of pooled YFV17D dIII monovalent sera (n=5) evaluated against each by 50% YFV17D plaque reduction neutralization (PRNT₅₀) assay on Vero cells using mouse immune ascites from YFV17D-immunized mice (MIAF) as a comparator. PRNT₅₀ titers were calculated by probit analysis.

### DETAILED DESCRIPTION OF THE INVENTION

The present invention relates to a novel Flavivirus subunit vaccine, its use, and its methods of manufacture. The Flavivirus vaccine is exemplified by a multivalent Dengue virus vaccine of the present invention, a monovalent Yellow Fever virus vaccine of the present invention, and a multivalent combined Dengue virus/Yellow Fever virus vaccine of the present invention. However, these exemplary vaccine formulations confirm that the invention can be practiced using any Flavivirus E polypeptide domain III (dIII).

One Dengue subunit vaccine of the present invention is a tetravalent vaccine that includes a Dengue dIII polypeptide for each of Dengue serotypes 1-4 (DEN1 to DEN4). In one embodiment, the vaccine is one that is capable of inducing, upon administration to a subject, a neutralizing antibody response against each of DEN1 to DEN4 that exceeds PRNT₅₀ value of 150. In another embodiment, the vaccine is one that is capable of inducing, upon administration to a subject, a neutralizing antibody response against each of DEN1 to DEN4 that exceeds PRNT₅₀ value of 200.

The Dengue dIII polypeptides of the present invention are preferably utilized with few, if any, amino acids from associated dI or dII fragments of the Dengue E polyprotein. In certain embodiments, up to 5 or up 10 amino acids wholly or partly from other E protein domains can be present on the N- or C-terminal ends of the dIII polypeptides of the present invention. In other embodiments, the Dengue dIII polypeptides are preferably entirely free of dI or dII polypeptide domains, and consist of no additional E protein epitopes that lie outside of dIII.

The DEN1 dIII polypeptide can have any known or hereafter isolated sequence of a DEN1 viral isolate. Preferably, the DEN1 dIII polypeptide has an amino acid sequence according to consensus SEQ ID NO: 1 as follows:

KGMSYVMCTG SFKLEKEVAE TQHGTVLVQX KYEGTDAPCK
IPFSTQDEKG XTQNGRLITA NPIVTDKEKP VNIEAEPPFG
ESYIVXGAGE KALKLSWFKK

where each X at positions 30, 51, and 86 can be any amino acid. According to preferred embodiments of SEQ ID NO: 1, X at position 30 is V or I, X at position 51 is V, I, or A, and X at position 86 is V or I. Alternatively, other DEN1 dIII polypeptides preferably share at least 85%, 86%, 87%, 88%, or 89% identity to the consensus SEQ ID NO: 1 over its entire length, more preferably at least 90%, 91%, 92%, 93%, or 94% identity to the consensus SEQ ID NO: 1 over its entire length, and most preferably at least 95%, 96%, 97%, 98%, or 99% identity to the consensus SEQ ID NO: 1. Other embodiments of DEN1 dIII polypeptides can include deletions or additions of up to about 5 or up to about 10 amino acids at one or both of the ends of SEQ ID NO: 1 or its homologs.

Exemplary DEN1 dIII polypeptides include, without limitation, the dIII polypeptide sequences of SEQ ID NOS: 2-6 illustrated in FIG. 1. The nucleic acid molecules (DNA or RNA) encoding each of these DEN1 dIII polypeptides can be identified using the Genbank Accession Nos. identified in the Figure legend. A comparison of SEQ ID NOS: 2-6 along with the consensus SEQ ID NO: 1 is illustrated in the ClustalW multiple sequence alignment of FIG. 1.

The DEN2 dIII polypeptide can have any known or hereafter isolated sequence of a DEN2 viral isolate. Preferably, the DEN2 dIII polypeptide has an amino acid sequence according to consensus SEQ ID NO: 7 as follows:

KGMSYSMCTG KFKXVKEIAE TQHGTIVXRV QYEGDGSPCK
IPFEIXDLEK RHVLGRLITV NPIVTEKDSP XNIEAEPPFG
DSYXIIGVEP GQLKLNWFKK

where each X at positions 14, 28, 46, 71, and 84 can be any amino acid. According to preferred embodiments of SEQ ID NO: 7, X at position 14 is V or I, X at position 28 is V or I, X at position 46 is M or T, X at position 71 is V or I, and X at position 84 is V or I. Alternatively, other DEN2 dIII polypeptides preferably share at least 85%, 86%, 87%, 88%, or 89% identity to the consensus SEQ ID NO: 7 over its entire length, more preferably at least 90%, 91%, 92%, 93%, or 94% identity to the consensus SEQ ID NO: 7 over its entire length, and most preferably at least 95%, 96%, 97%, 98%, or 99% identity to the consensus SEQ ID NO: 7. Other embodiments of DEN2 dIII polypeptides can include deletions or additions of up to about 5 or up to about 10 amino acids at one or both of the ends of SEQ ID NO: 7 or its homologs.

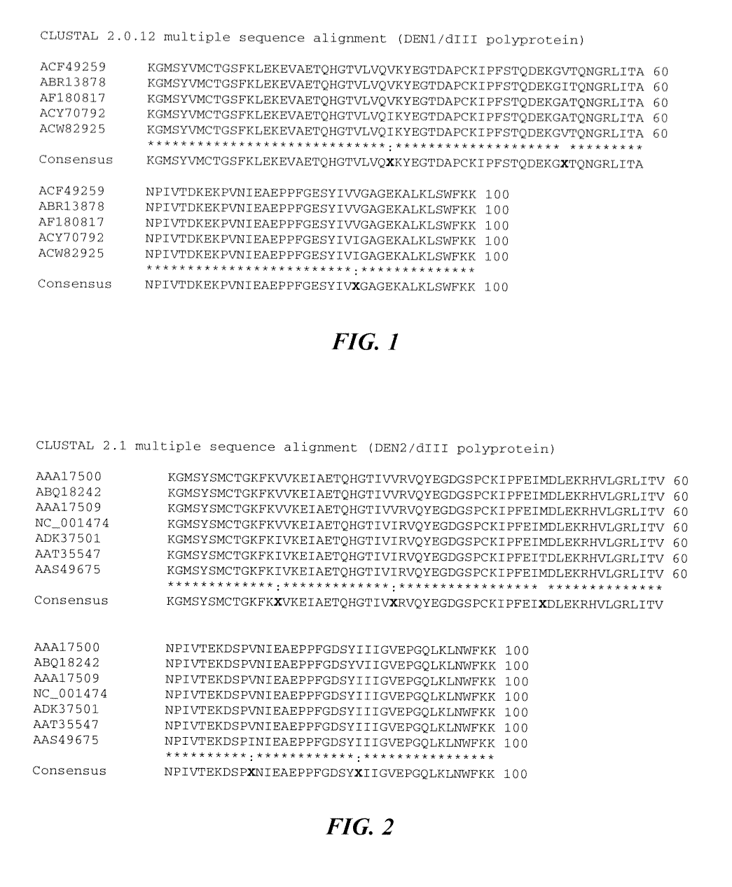

Exemplary DEN2 dIII polypeptides include, without limitation, the polypeptide sequences of SEQ ID NOS: 8-14 illustrated in FIG. 2. The nucleic acid molecules (DNA or RNA) encoding each of these DEN2 dIII polypeptides can be identified using the Genbank Accession Nos. identified in the Figure legend. A comparison of SEQ ID NOS: 8-14 along with the consensus SEQ ID NO: 7 is illustrated in the ClustalW multiple sequence alignment of FIG. 2.

The DEN3 dIII polypeptide can have any known or hereafter isolated sequence of a DEN3 viral isolate. Preferably, the DEN3 dIII polypeptide has an amino acid sequence according to consensus SEQ ID NO: 15 as follows:

XGMSYAMCLN TFVLKKEVSE TQHGTXLIKV EYKGEDAPCK
IPFSTEDGQG KAHNGRLITA NPVVTKKEEP VNIEAEPPFG
ESNIVIGXGD KALKINWYXX

where each X at positions 1, 26, 88, 99, and 100 can be any amino acid. According to preferred embodiments of SEQ ID NO: 15, X at position 1 is K or R, X at position 26 is L or I, X at position 88 is I or V, X at position 99 is K or R, and X at position 100 is K or R. Alternatively, other DEN3 dIII polypeptides preferably share at least 85%, 86%, 87%, 88%, or 89% identity to the consensus SEQ ID NO: 15 over its entire length, more preferably at least 90%, 91%, 92%, 93%, or 94% identity to the consensus SEQ ID NO: 15 over its entire length, and most preferably at least 95%, 96%, 97%, 98%, or 99% identity to the consensus SEQ ID NO: 15. Other embodiments of DEN3 dIII polypeptides can include deletions or additions of up to about 5 or up to about 10 amino acids at one or both of the ends of SEQ ID NO: 15 or its homologs.

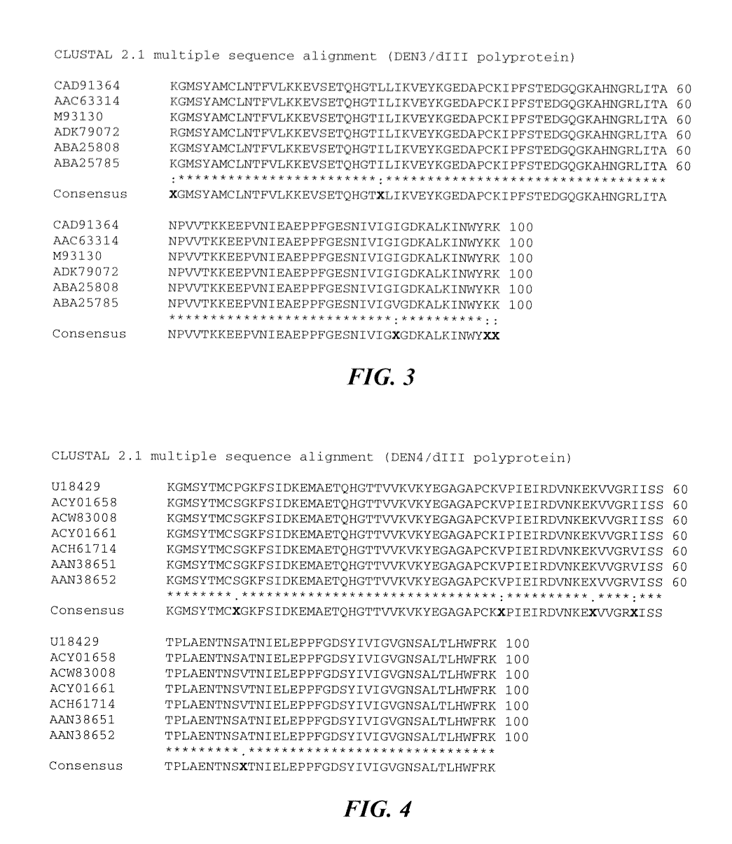

Exemplary DEN3 dIII polypeptides include, without limitation, the polypeptide sequences of SEQ ID NOS: 16-21 illustrated in FIG. 3. The nucleic acid molecules (DNA or RNA) encoding each of these DEN3 dIII polypeptides can also be identified using the Genbank Accession Nos. identified in the Figure legend. A comparison of SEQ ID NOS: 16-21 along with the consensus SEQ ID NO: 15 is illustrated in the ClustalW multiple sequence alignment of FIG. 3.

The DEN4 dIII polypeptide can have any known or hereafter isolated sequence of a DEN4 viral isolate. Preferably, the DEN4 dIII polypeptide has an amino acid sequence according to consensus SEQ ID NO: 22 as follows:

KGMSYTMCXG KFSIDKEMAE TQHGTTVVKV KYEGAGAPCK
XPIEIRDVNK EKVVGRXISS TPLAENTNSX TNIELEPPFG
DSYIVIGVGN SALTLHWFRK

where each X at positions 9, 41, 57, and 60 can be any amino acid. According to preferred embodiments of SEQ ID NO: 22, X at position 9 is S or P, X at position 41 is V or I, X at position 57 is V or I, and X at position 60 is A or V. Alternatively, other DEN4 dIII polypeptides preferably share at least 85%, 86%, 87%, 88%, or 89% identity to the consensus SEQ ID NO: 22 over its entire length, more preferably at least 90%, 91%, 92%, 93%, or 94% identity to the consensus SEQ ID NO: 22 over its entire length, and most preferably at least 95%, 96%, 97%, 98%, or 99% identity to the consensus SEQ ID NO: 22. Other embodiments of DEN4 dIII polypeptides can include deletions or additions of up to about 5 or up to about 10 amino acids at one or both of the ends of SEQ ID NO: 22 or its homologs.

Exemplary DEN4 dIII polypeptides include, without limitation, the polypeptide sequences of SEQ ID NOS: 23-29 illustrated in FIG. 4. The nucleic acid molecules (DNA or RNA) encoding each of these DEN4 dIII polypeptides can also be identified using the Genbank Accession Nos. identified in the Figure legend. A comparison of SEQ ID NOS: 23-29 along with the consensus SEQ ID NO: 22 is illustrated in the ClustalW multiple sequence alignment of FIG. 4.

The YFV dIII polypeptide can have any known or hereafter isolated sequence of a YFV isolate. Preferably, the YFV dIII polypeptide has an amino acid sequence according to consensus SEQ ID NO: 30 as follows:

KGTSYKXCTD KMXFVKNPTD TXHGTXVMQV KVXKGAPCXI
PVXVADDLTA XXNKGILVTV NXIASTNXDE VLIEVNPPFG
DSYIIXGXGD SRLTYQWHKE

where each X at positions 7, 13, 22, 26, 33, 39, 43, 51, 52, 62, 68, 85, and 87 can be any amino acid. According to preferred embodiments of SEQ ID NO: 30, X at position 7 is M or I, X at position 13 is S or F, X at position 22 is G or D, X at position 26 is A or V, X at position 33 is P or S, X at position 39 is K, R, or G, X at position 43 is M or I, X at position 51 is A or S, X at position 52 is V or I, X at position 62 is P or S, X at position 68 is D or E, X at position 85 is V or I, and X at position 87 is T or R. Alternatively, other YFV dIII polypeptides preferably share at least 85%, 86%, 87%, 88%, or 89% identity to the consensus SEQ ID NO: 30 over its entire length, more preferably at least 90%, 91%, 92%, 93%, or 94% identity to the consensus SEQ ID NO: 30 over its entire length, and most preferably at least 95%, 96%, 97%, 98%, or 99% identity to the consensus SEQ ID NO: 30. Other embodiments of YFV dIII polypeptides can include deletions or additions of up to about 5 or up to about 10 amino acids at one or both of the ends of SEQ ID NO: 30 or its homologs.

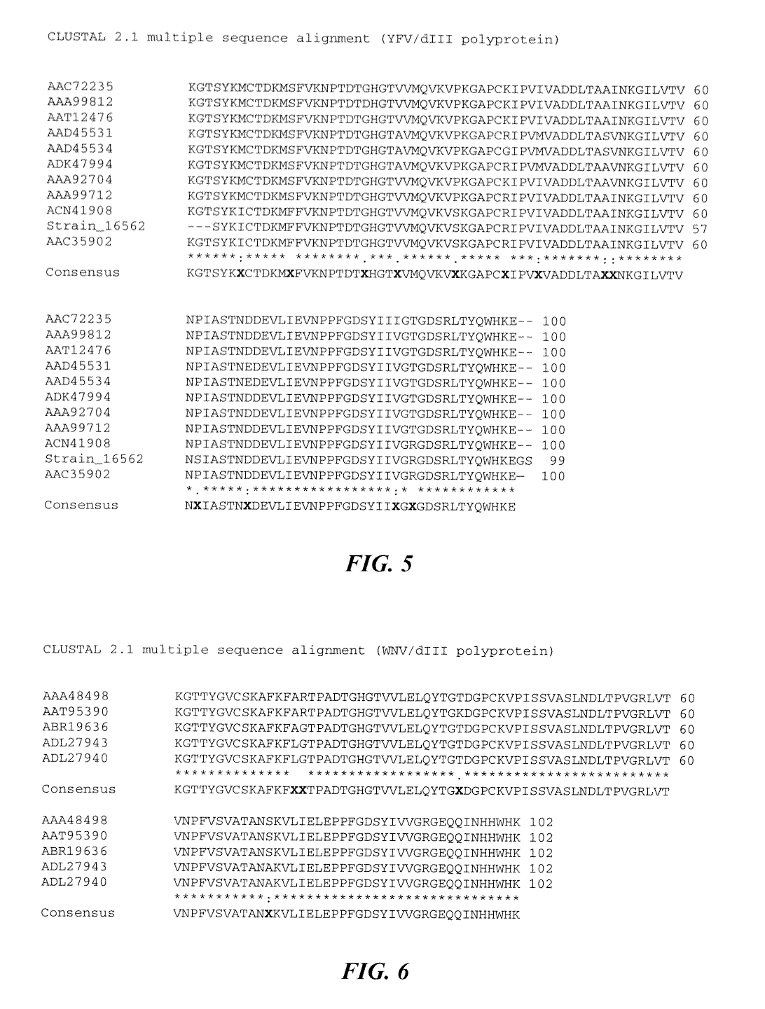

Exemplary YFV dIII polypeptides include, without limitation, the polypeptide sequences of SEQ ID NOS: 31-41 illustrated in FIG. 5. The nucleic acid molecules (DNA or RNA) encoding each of these YFV dIII polypeptides can also be identified using the Genbank Accession Nos. identified in the Figure legend. A comparison of SEQ ID NOS: 31-41 along with the consensus SEQ ID NO: 30 is illustrated in the ClustalW multiple sequence alignment of FIG. 5.

The WNV dIII polypeptide can have any known or hereafter isolated sequence of a WNV isolate. Preferably, the WNV dIII polypeptide has an amino acid sequence according to consensus SEQ ID NO: 42 as follows:

KGTTYGVCSK AFKFXXTPAD TGHGTVVLEL QYTGXDGPCK
VPISSVASLN DLTPVGRLVT VNPFVSVATA NXKVLIELEP
PFGDSYIVVG RGEQQINHHW HK

where each X at positions 15, 16, 35, and 72 can be any amino acid. According to preferred embodiments of SEQ ID NO: 42, X at position 15 is L or A, X at position 16 is R or G, X at position 35 is T or K, and X at position 72 is S or A. Alternatively, other WNV dIII polypeptides preferably share at least 85%, 86%, 87%, 88%, or 89% identity to the consensus SEQ ID NO: 42 over its entire length, more preferably at least 90%, 91%, 92%, 93%, or 94% identity to the consensus SEQ ID NO: 42 over its entire length, and most preferably at least 95%, 96%, 97%, 98%, or 99% identity to the consensus SEQ ID NO: 42. Other embodiments of WNV dIII polypeptides can include deletions or additions of up to about 5 or up to about 10 amino acids at one or both of the ends of SEQ ID NO: 42 or its homologs.

Exemplary WNV dIII polypeptides include, without limitation, the polypeptide sequences of SEQ ID NOS: 43-47 illustrated in FIG. 6. The nucleic acid molecules (DNA or RNA) encoding each of these WNV dIII polypeptides can also be identified using the Genbank Accession Nos. identified in the Figure legend. A comparison of SEQ ID NOS: 43-47 along with the consensus SEQ ID NO: 42 is illustrated in the ClustalW multiple sequence alignment of FIG. 6.

The JEV dIII polypeptide can have any known or hereafter isolated sequence of a JEV isolate. Preferably, the JEV dIII polypeptide has an amino acid sequence according to consensus SEQ ID NO: 48 as follows:

KGTTYGMCTX KFSFAKNPAD TGHGTVVIEL SYXGXDGPCK
IPIVSXASLN DXTPXGRLVT VNPFVATSSA NSKVLVEMEP
PFGDSYIVVG RXXKQINHHW HK

where each X at positions 10, 33, 35, 46, 52, 55, 92, and 93 can be any amino acid. According to preferred embodiments of SEQ ID NO: 48, X at position is 10 is K, E, or G, X at position 33 is S or C, X at position 35 is S or R, X at position 46 is V or A, X at position 52 is M or L, X at position 55 is A or V, X at position 92 is G or E, and X at position 93 is D or N. Alternatively, other JEV dIII polypeptides preferably share at least 85%, 86%, 87%, 88%, or 89% identity to the consensus SEQ ID NO: 48 over its entire length, more preferably at least 90%, 91%, 92%, 93%, or 94% identity to the consensus SEQ ID NO: 48 over its entire length, and most preferably at least 95%, 96%, 97%, 98%, or 99% identity to the consensus SEQ ID NO: 48. Other embodiments of JEV dIII polypeptides can include deletions or additions of up to about 5 or up to about 10 amino acids at one or both of the ends of SEQ ID NO: 48 or its homologs.

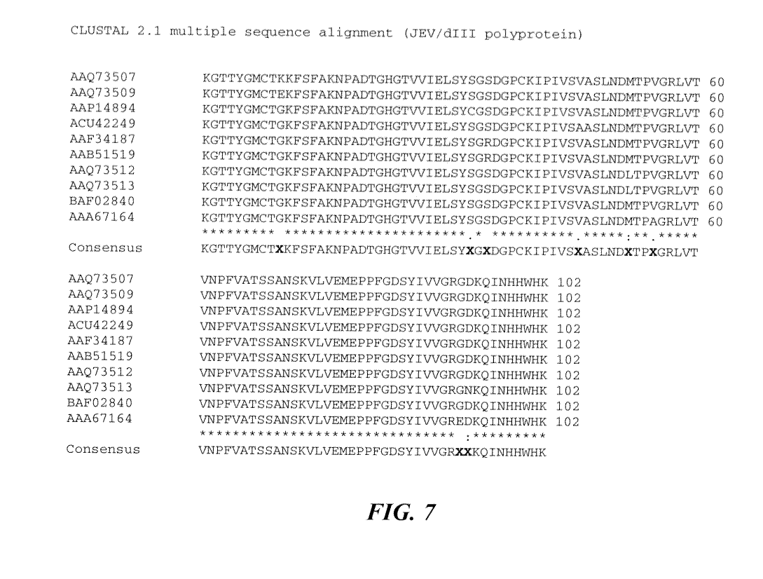

Exemplary JEV dIII polypeptides include, without limitation, the polypeptide sequences of SEQ ID NOS: 49-58 illustrated in FIG. 7. The nucleic acid molecules (DNA or RNA) encoding each of these WNV dIII polypeptides can also be identified using the Genbank Accession Nos. identified in the Figure legend. A comparison of SEQ ID NOS: 49-58 along with the consensus SEQ ID NO: 48 is illustrated in the ClustalW multiple sequence alignment of FIG. 7.

As discussed hereinafter, the dIII polypeptides of the present invention can also include a polypeptide sequence useful for purification, such as a polyhistidine (e.g., His₆) tag that can be used for affinity purification of the dIII polypeptide; a residue or amino acid sequence useful for linking the dIII polypeptide to another protein or polypeptide; or a residue or amino acid sequence that is an artifact of cloning procedures used to construct the recombinant expression system used to express the polypeptide. The polyhistidine residues can be linked to one of the N- or C-terminals, the latter being demonstrated in the accompanying Examples.

A further aspect of the present invention relates to a fusion protein including any one of the isolated dIII polypeptide fragments of the present invention.

In one embodiment, the fusion protein includes one of the isolated dIII polypeptide fragments described supra linked by an in-frame fusion to an adjuvant polypeptide.

By way of example, and without limitation, suitable fusion proteins of the present invention include an adjuvant polypeptide fused in-frame to any one of the above listed DEN1 dIII polypeptides (e.g., SEQ ID NOS: 1-6). Suitable fusion proteins of the present invention may also include an adjuvant polypeptide fused in-frame to any one of the above listed DEN2 dIII polypeptides (e.g., SEQ ID NOS: 7-14), to any one of the above listed DEN3 dIII polypeptides (e.g., SEQ ID NOS: 15-21), or to any one of the above listed DEN4 dIII polypeptides (e.g., SEQ ID NOS: 22-29). Other suitable fusion proteins of the present invention include an adjuvant polypeptide fused in-frame to any one of the above listed YFV dIII polypeptides (e.g., SEQ ID NOS: 30-41), to any one of the above listed WNV dIII polypeptides (e.g., SEQ ID NOS: 42-47), or to any one of the above listed JEV dIII polypeptides (e.g., SEQ ID NOS: 48-58). The adjuvant polypeptide can be any peptide adjuvant known in art including, but not limited to, flagellin, human papillomavirus (HPV) L1 or L2 proteins (see PCT International Pat. Pub. WO99/61052 to Rose et al. and PCT International Pat. Pub. WO94/20137 to Rose et al., both of which are hereby incorporated by reference in their entirety), herpes simplex glycoprotein D (gD), complement C4 binding protein, toll-like receptor-4 (TLR4) ligand, and IL-1β. The dIII polypeptides are preferably joined to the adjuvant polypeptide with a flexible linker region, which should allow the dIII and adjuvant polypeptides to fold properly.

In an alternative embodiment, two or more dIII polypeptides can be presented as a single fusion protein with or without an adjuvant polypeptide. Thus, for example, in a Dengue vaccine, the dIII polypeptides for DEN1 dIII, DEN2 dIII, DEN3 dIII, and DEN4 dIII can be linked together as a single molecule. In another embodiment of the Dengue vaccine, the dIII polypeptides for any two of DEN1 dIII, DEN2 dIII, DEN3 dIII, and DEN4 dIII can be linked together as a single molecule and the dIII polypeptides for the remaining two of DEN1 dIII, DEN2 dIII, DEN3 dIII, and DEN4 dIII can be linked together as a separate molecule, both of which would be included in the same vaccine formulation. The dIII polypeptides are preferably joined together with a flexible linker region, described supra, which should allow the individual dIII polypeptides to fold properly. Such hybrid fusion proteins can also be linked to an adjuvant polypeptide as described above.

The dIII fusion proteins of the present invention (e.g., containing DEN1 dIII, DEN2 dIII, DEN3 dIII, DEN4 dIII, YFV dIII, WNV dIII, or JEV dIII) can be generated using standard techniques known in the art. For example, the fusion polypeptide can be prepared by translation of an in-frame fusion of the polynucleotide sequences encoding the dIII and the adjuvant as well as any purification tag, i.e., a hybrid gene. The hybrid gene encoding the fusion polypeptide is inserted into an expression vector which is used to transform or transfect a host cell. Alternatively, the polynucleotide sequence encoding the dIII polypeptide is inserted into an expression vector in which the polynucleotide encoding the adjuvant is already present.

The peptide adjuvant of the fusion protein can be fused to the N- or C-terminal end of the dIII polypeptide. Fusions between the dIII polypeptide and the protein adjuvant may be such that the amino acid sequence of the dIII polypeptide is directly contiguous with the amino acid sequence of the adjuvant.

Alternatively, the dIII portion may be coupled to the adjuvant by way of a short linker sequence. Suitable linker sequences include glycine rich linkers (e.g., GGGS₂₋₃), serine-rich linkers (e.g., GS_{N}), or other flexible immunoglobulin linkers as disclosed in U.S. Pat. No. 5,516,637 to Huang et al, which is hereby incorporated by reference in its entirety.

With respect to the HPV-L1 or L2 fusion proteins, it is desirable that the L1 or L2 proteins be capable of self-assembly in the form of a virus-like particle or capsomere that includes the dIII polypeptide as a surface exposed region (so as to afford a neutralizing response against the dIII polypeptide). It is well established that the HPV L1 capsomeres and VLPs are immunogenic and behave as an adjuvant.

Papillomaviruses are small, double-stranded, circular DNA tumor viruses. The papillomavirus virion shells contain the L1 major capsid protein and the L2 minor capsid protein and the L2 minor capsid protein. Expression of L1 protein alone or in combination with L2 protein in eukaryotic or prokaryotic expression systems is known to result in the assembly of capsomeres and VLPs.

As used herein, the term “capsomere” is intended to mean a pentameric assembly of papillomavirus L1-containing fusion polypeptides. Native L1 capsid proteins self-assemble via intermolecular disulfide bonds to form pentamers (capsomeres). It has been shown previously that L1 capsomeres induce serotype-specific neutralizing antibodies in mice, induce L1-specific CTL responses and tumor regression in mice, and that the vast majority of surface-exposed anti-HPV antibody epitopes are located on capsomere loops (Rose et al., “Human Papillomavirus Type 11 Recombinant L1 Capsomeres Induce Virus-Neutralizing Antibodies,” J Virol 72:6151-6154 (1998); Ohlschlager et al., “Human Papillomavirus Type 16 L1 Capsomeres Induce L1-specific Cytotoxic T Lymphocytes and Tumor Regression in C57BL/6 Mice,” J Virol. 77: 4635-4645 (2003); and Yuan et al., “Immunization with a Pentameric L1 Fusion Protein Protects against Papillomavirus Infection,” J. Virol 75: 7843-7853 (2001), each of which is hereby incorporated by reference in its entirety). Taken together, capsomeres have the potential as a vaccine platform to elicit a broad range of cellular and humoral immune responses.

As used herein, the term “virus-like particle” or VLP is intended to mean a particle comprised of a higher order assembly of capsomeres. VLPs are non-infectious and non-replicating, yet morphologically similar to native papillomavirus virion. One example of such a higher order assembly is a particle that has the visual appearance of a whole (72 capsomere) or substantially whole, empty papillomavirus capsid, which is about 50 to about 60 nm in diameter and has a T=7 icosahedral construction. Another example of such a higher order assembly is a particle of about 30 to about 35 nm in diameter, which is smaller than the size of a native papillomavirus virion and has a T=1 construction (containing 12 capsomeres). For purposes of the present invention, other higher order assemblies of capsomeres are also intended to be encompassed by the term VLP. The VLPs and capsomeres preferably, but need not, replicate conformational epitopes of the native papillomavirus from which the L1 protein or polypeptide or L2 protein or polypeptide is derived. Methods for assembly and formation of human papillomavirus VLPs and capsomeres of the present invention are well known in the art (U.S. Pat. No. 6,153,201 to Rose et al.; U.S. Pat. No. 6,165,471 to Rose et al.; WO 94/020137 to Rose et al., each of which is hereby incorporated by reference in its entirety).

As used herein, the term “chimeric” is intended to denote VLPs or capsomeres that include polypeptide components from two or more distinct sources (e.g., a papillomavirus and a dIII polypeptide of the type described above). This term is not intended to confer any meaning concerning the specific manner in which the polypeptide components are bound or attached together.

In one embodiment, the chimeric papillomavirus VLP or capsomere includes an L1 polypeptide and, optionally, an L2 polypeptide, and a dIII protein or polypeptide fragment thereof that includes a first epitope, where the dIII protein or polypeptide fragment thereof is attached to one or both of the L1 and L2 polypeptides.

The L1 polypeptide can be full-length L1 protein or an L1 polypeptide fragment. According to one embodiment, the full-length L1 protein or L1 polypeptide fragment can be VLP assembly-competent (that is, the L1 polypeptide will self-assemble to form capsomeres that are competent for self-assembly into a higher order assemblies, thereby forming a VLP). According to another embodiment, the full-length L1 protein or L1 polypeptide fragment can be VLP assembly-incompetent (that is, the L1 polypeptide will form capsomeres that are unable to assemble into higher order assemblies of a VLP). L1 polypeptides that lack at least a portion of the helix 4 (“h4”) domain, preferably the entire h4 domain (residues 412-428 of HPV-16 L1) and its surrounding amino acids, also lack the ability to form L1 VLPs, but the resulting L1 derivatives are capable of self-assembly into capsomeres (Bishop et al., “Structure-based Engineering of Papillomavirus Major Capsid L1: Controlling Particle Assembly,” Virol J 4:3, pp. 1-6 (2007), which is hereby incorporated by reference in its entirety).

The L1 sequences are known for substantially all papillomavirus genotypes identified to date, and any of these L1 sequences or fragments can be employed in the present invention. Examples of L1 polypeptides include, without limitation, full-length L1 polypeptides, L1 truncations that lack the native C-terminus, L1 truncations that lack the native N-terminus, and L1 truncations that lack an internal domain. As described hereinafter, L1 fusion proteins can include the heterologous, dIII polypeptide linked at the N-terminus of the L1 polypeptide, the C-terminus of the L1 polypeptide, or at internal sites of the L1 polypeptide, including where portions of the native L1 sequence have been deleted.

The L2 polypeptide can be full-length L2 protein or an L2 polypeptide fragment. The L2 sequences are known for substantially all papillomavirus genotypes identified to date, and any of these L2 sequences or fragments can be employed in the present invention. Examples of L2 polypeptides include, without limitation, full-length L2 polypeptides, L2 truncations that lack the native C-terminus, L2 truncations that lack the native N-terminus, and L2 truncations that lack an internal domain. As described hereinafter, L2 fusion proteins can include the heterologous, dIII polypeptide linked at the N-terminus of the L2 polypeptide, the C-terminus of the L2 poly peptide, or at internal sites of the 12 polypeptide, including where portions of the native L2 sequence have been deleted.

The chimeric papillomavirus VLPs and capsomeres can be formed using the L1 and optionally L2 polypeptides from any animal papillomavirus, or derivatives or fragments thereof. Thus, any known (or hereafter identified) L1 and optional L2 sequences of human, bovine, equine, ovine, porcine, deer, canine, feline, rodent, rabbit, etc., papillomaviruses can be employed to prepare the VLPs or capsomeres of the present invention.

In one embodiment of the present invention, the L1 and optionally L2 polypeptides of the papillomavirus VLP are derived from human papillomaviruses. Preferably, they are derived from HPV-6, HPV-11, HPV-16, HPV-18, HPV-31, HPV33, HPV-35, HPV-39, HPV-45, HPV-52, HPV-54, HPV-58, HPV-59, HPV-64, or HPV-68. For a near complete listing of papillomavirus genotypes and their relatedness, see de Villiers et al., “Classification of Papillomaviruses,” Virology 324:17-27 (2004), which is hereby incorporated by reference in its entirety. The L1 and L2 sequences are known for substantially all papillomaviruses identified to date, e.g., HPV-18 (Genbank accessions NC_{—}001357 and X05015, which are hereby incorporated by reference in its entirety); HPV-64 (NC_{—}001676 and U37488, which are hereby incorporated by reference in its entirety); and all other HPV genotypes. Exemplary genital-specific genotypes of human papillomavirus include, but are not limited to HPV-6, -11, -16, -18, -30, -31, -33, -34, -35, -39, -60, -62, -43, -64, -65, -51, -52, -53, -54, -56, -58, -59, -61, -62, -66, -67, -68, -69, -70, -71, -74, -81, -85, -86, -87, -89, -90, -91, -92, -101, -102, -103, and -106. Some of the genital-specific genotype human papillomaviruses are associated with cancer, including HPV-16, -18, -31, -33, -35, -39, -45, -51, -52, -56, -58, -59, -66, -67, -68, -73, and -82. Exemplary nongenital-specific genotypes of human papillomavirus include, but are not limited to, HPV-1, -2, -3, -4, -7, -10, -22, -28, -29, -36, -37, -38, -41, -48, -49, -60, -63, -67, -72, -76, -77, -80, -88, -92, -93, -94, -98, -95, -96, and -107. VLPs or capsomeres of other HPV genotypes, whether newly discovered or previously known, can also be used.

According to one embodiment of the present invention, the dIII protein or polypeptide fragment is attached via an in-frame gene fusion to one or both of the L1 and L2 polypeptides such that recombinant expression of the L1 and/or L2 fusion protein results in incorporation of the dIII protein or polypeptide into the self-assembled capsomere or VLP's of the present invention (i.e., with the epitopes thereof available for inducing the elicitation of a high-titer neutralizing antibody response).

By way of example, and without limitation, suitable L1-dIII fusion proteins include full length L1 polypeptides fused in-frame to one of the above-listed dIII polypeptides (e.g. DEN dIII polypeptides (SEQ ID NOS: 1-29)); truncated N-terminal L1 polypeptides fused in-frame to one of the above listed dIII polypeptides; truncated C-terminal L1 polypeptides (lacking amino acid residues 2-8, i.e., residues SLWLPSE of HPV-16 L1 polypeptides) fused in-frame to one of the above-listed dIII polypeptides; L1 polypeptides having an h4-domain deletion and one of the above-listed dIII polypeptides polypeptides inserted at the h4-deletion site; full length L2 polypeptides fused in-frame to one of the above-listed dIII polypeptides polypeptides; truncated N-terminal L2 polypeptides fused in-frame to one of the above-listed dIII polypeptides polypeptides; and truncated C-terminal L2 polypeptides fused in-frame to one of the above-listed dIII polypeptides polypeptides.

In addition to these fusion proteins, L1 or L2 polypeptides can be joined in-frame with multiple dIII polypeptides containing different epitopes. For example, the L1 or L2 full-length, N-terminal, or C-terminal polypeptides can be linked in-frame to a first dIII polypeptide containing a first epitope (or more) and a second dIII polypeptide containing a second epitope (or more). Alternatively, both L1-dIII fusion polypeptides and L2-dIII fusion polypeptides can be prepared and expressed for co-assembly, whereby the two fusion proteins contain the same or, more preferably, distinct dIII epitopes. Regardless of the approach for introducing multiple dIII epitopes into the capsomeres or VLPs of the invention, both the first and second epitopes are preferably neutralizing epitopes. In this way, it is possible to use the capsomeres or VLPs to generate a protective immune response that is not dedicated to a single dIII epitope.

The making of VLPs and capsomeres according to this embodiment basically involves the preparation of recombinant genetic constructs using known procedures, followed by the expression of the genetic constructs in recombinant host cells, and then the isolation and purification of the self-assembled VLPs and/or capsomeres.

The genetic constructs encoding the full or partial length L1 polypeptide, full or partial length L2 polypeptide, L1 polypeptide/dIII polypeptide fusion proteins, and L2 polypeptide/dIII polypeptide fusion proteins, can be prepared according to standard recombinant procedures. Basically, DNA molecules encoding the various polypeptide components of the fusion protein (to be prepared) are ligated together to form an in-frame gene fusion that results in, for example, a single open reading frame that expresses a single fusion protein including the papillomavirus capsid polypeptide (L1 or L2) fused to the dIII polypeptide. The DNA coding sequences, or open reading frames, encoding the whole or partial L1 and/or L2 polypeptides and/or fusion proteins can be ligated to appropriate regulatory elements that provide for expression (i.e., transcription and translation) of the fusion protein encoded by the DNA molecule. These regulatory sequences, typically promoters, enhancer elements, transcription terminal signals, etc., are well known in the art for various express systems.

When a prokaryotic host cell is selected for subsequent transformation, the promoter region used to construct the recombinant DNA molecule (i.e., transgene) should be appropriate for the particular host. As is well known in the art, the DNA sequences of eukaryotic promoters, for expression in eukaryotic host cells, differ from those of prokaryotic promoters. Eukaryotic promoters and accompanying genetic signals may not be recognized in or may not function in a prokaryotic system, and, further, prokaryotic promoters are not recognized and do not function in eukaryotic cells.

Thus, the DNA molecules encoding the polypeptide products to be expressed in accordance with the present invention can be cloned into a suitable expression vector using standard cloning procedures known in the art, including restriction enzyme cleavage and ligation with DNA ligase as described by Sambrook et al., Molecular Cloning: A Laboratory Manual, Second Edition, Cold Spring Harbor Press, NY (2001) and Ausubel et al., Current Protocols in Molecular Biology, John Wiley & Sons, New York, N.Y. (2008), both of which are hereby incorporated by reference in their entirety. Recombinant molecules, including plasmids, can be introduced into cells via transformation, particularly transduction, conjugation, mobilization, or electroporation. Once these recombinant plasmids are introduced into unicellular cultures, including prokaryotic organisms and eukaryotic cells, the cells are grown in tissue culture and vectors can be replicated.

For the recombinant expression of papillomavirus L1 and/or L2 fusion proteins, and resulting capsomere and/or VLP assembly, the recombinant vectors produced above are used to infect a host cell. Any number of vector-host combinations can be employed, including plant cell vectors (Agrobacterium) and plant cells, yeast vectors and yeast hosts, baculovirus vectors and insect host cells, vaccinia virus vectors and mammalian host cells, or plasmid vectors in E. coli. Additional mammalian expression vectors include those derived from adenovirus adeno-associated virus, nodavirus, and retroviruses.

In one embodiment, the capsomeres and/or VLPs of the present invention are formed in Sf-9 insect cells upon expression of the L1 and optionally L2 proteins or polypeptides using recombinant baculovirus. General methods for handling and preparing baculovirus vectors and baculovirus DNA, as well as insect cell culture procedures, are outlined in The Molecular Biology of Baculoviruses, Doerffer et al., Eds. Springer-Verlag, Berlin, pages 31-49; Kool et al., “The Structural and Functional Organization of the Autographa californica Nuclear Polyhedrosis Virus Genome,” Arch. Virol. 130:1-16 (1993); Kimbauer et al., “Efficient Self-assembly of Human Papillomavirus Type 16 L1 and L1-L2 into Virus-like Particles,” J. Virol. 67(12):6929-6936 (1993); Volpers et al., “Binding and Internalization of Human Papillomavirus Type 33 Virus-like Particles by Eukaryotic Cells,” J. Virol. 69:3258-3264 (1995); Rose et al., “Expression of Human Papillomavirus Type 11 L1 Protein in Insect Cells: in vivo and in vitro Assembly of Viruslike Particles,” J Virol. 67(4):1936-1944 (1993), all of which are hereby incorporated by reference in their entirety).

However, recombinant expression vectors and regulatory sequences suitable for expression of papillomavirus polypeptides in yeast or mammalian cells are well known and can be used in the present invention (see Hagensee et al., “Self-assembly of Human Papillomavirus Type 1 Capsids by Expression of the L1 Protein Alone or by Coexpression of the L1 and L2 Capsid Proteins,” J. Virol. 67(1):315-22 (1993); Sasagawa et al., “Synthesis and Assembly of Virus-like Particles of Human Papillomaviruses Type 6 and Type 16 in Fission Yeast Schizosaccharomyces pombe,” Virology 2016:126-195 (1995); Buonamassa et al., “Yeast Coexpression of Human Papillomavirus Types 6 and 16 Capsid Proteins,” Virol. 293(2):335-344 (2002); U.S. Pat. No. 7,112,330 to Buonamassa et al.; U.S. Patent Publ. No. 20080166371 to Jansen et al., all of which are hereby incorporated by reference in their entirety).

Regardless of the host-vector system utilized for the recombinant expression and self-assembly of capsomeres and/or VLPs, these products can be isolated from the host cells, and then purified using known techniques. For example, the purification of the VLPs or capsomeres can be achieved very simply by means of centrifugation in CsCl or sucrose gradients (Kirnbauer et al., “Efficient Self-assembly of Human Papillomavirus Type 16 L1 and L1-L2 into Virus-like Particles,” J Virol. 67(12): 6929-6936 (1993); Sasagawa et al., “Synthesis and Assembly of Virus-like Particles of Human Papillomaviruses Type 6 and Type 16 in Fission Yeast Schizosaccharomyces pombe,” Virology 2016:126-195 (1995); Volpers et al., “Binding and Internalization of Human Papillomavirus Type 33 Virus-like Particles by Eukaryotic Cells,” J. Virol. 69:3258-3264 (1995); Rose et al., “Expression of Human Papillomavirus Type 11 L1 Protein in Insect Cells: in vivo and in vitro Assembly of Viruslike Particles,” J. Virol. 67(4): 1936-1944 (1993); Rose et al., “Serologic Differentiation of Human Papillomavirus (HPV) Types 11, 16, and 18 L1 Virus-like Particles (VLPs),” J. Gen. Virol., 75:2445-2449 (1994), each of which is hereby incorporated by reference in its entirety). Substantially pure VLP or capsomere preparations can be used as the active agent in a vaccine.

Alternatively, for expression in prokaryotes such as E. coli, a GST-fusion protein or other suitable chimeric protein can be expressed recombinantly, and thereafter purified and the GST portion cleaved to afford a self-assembly competent L1-dIII polypeptide that forms capsomeres or VLPs (see Chen et al., “Papillomavirus Capsid Protein Expression in Escherichia coli: Purification and Assembly of HPV11 and HPV16 L1,”J. Mol. Biol. 307:173-182 (2001), which is hereby incorporated by reference in its entirety). The resulting VLPs or capsomeres can be purified again to separate the structural assemblies from host cell by-products.

According to another embodiment of the present invention, non-chimeric, recombinant VLPs or capsomeres are first produced and purified, and then are thereafter modified by chemically conjugating the dIII polypeptide to the VLP or capsomere surface via small cross-linking molecules (Ionescu et al., “Pharmaceutical and Immunological Evaluation of Human Papillomavirus Virus Like Particle as an Antigen Carrier,”J. Pharm. Sci. 95:70-79 (2006), which is hereby incorporated by reference in its entirety). As a result of this conjugation, the resulting VLP or capsomere product is effectively decorated with anywhere from several hundred up to several thousand of the conjugated dIII polypeptide molecules per VLP (or corresponding amount per capsomere). This level of conjugation is capable of eliciting a strong, protective antibody response against the conjugated peptide sequence (Ionescu et al., “Pharmaceutical and Immunological Evaluation of Human Papillomavirus Virus Like Particle as an Antigen Carrier,”, J. Pharm. Sci. 95:70-79 (2006), which is hereby incorporated by reference in its entirety).

The dIII polypeptides can be conjugated with any suitable linker molecule, but preferably a hetero-bifunctional cross linker molecule. A number of hetero-bifunctional cross-linker molecules are known in the art, and are commercially available. Exemplary hetero-bifunctional crosslinker molecules include, without limitation, N-succinimidyl 3-(2-pyridyldithio)-propionate (“SPDP”), succinimidyl 6-10 (3-[2-pyridyldithio]-propionamido)hexanoate (“LC-SPDP”), sulfosuccinimidyl 4-[N-maleimidomethyl]cyclohexane-1-carboxylate (“Sulfo-SMCC”), succinimidyl-4-(N-maleimidomethyl)cyclohexane-1-carboxylate (“SMCC”), succinimidyl-4-[N-maleimidomethyl]cyclohexane-1-carboxy-[6-amidocaproate], [N-e-maleimidocaproyloxy]succinimide ester (“EMCS”), [N-e-maleimidocaproyloxy]sulfosuccinimide ester (“Sulfo-EMCS”), N-[g-maleimidobutyryloxy]succinimide ester (“GMBS”), N-[g-maleimidobutyryloxy]sulfosuccinimide ester (“Sulfo-GMBS”), N-[k-maleimidoundecanoyloxy]sulfosuccinimide ester (“Sulfo-KMUS”), 4-succinimidyloxycarbonyl-methyl-a-[2-pyridyldithio]toluene (“SMPT”), 4-sulfosuccinimidyl-6-methyl-a-(2-pyridyldithio)toluamido]hexanoate (“Sulfo-LC-SMPT”), m-maleimidobenzoyl-N-hydroxysuccinimide ester (“MBS”), m-maleimidobenzoyl-N-hydroxysulfosu ccinimide ester (“Sulfo-MBS”), Nsuccinimidyl[4-iodoacetyl]aminobenzoate (“SIAB”), N-sulfosuccinimidyl[4-iodoacetyl]aminobenzoate (“Sulfo-SIAB”), succinimidyl 4-[p-maleimidophenyl]butyrate (“SMPB”), sulfosuccinimidyl 4-[p-maleimidophenyl]butyrate (“Sulfo-SMPB”), N-a-maleimidoacetoxy) succinimide ester (“AMAS”), N-[4-(p-azidosalicylamido) butyl]-3′-(2′-pyridyldithio)propionamide (“APDP”), N-[β-maleimidopropyloxy]succinimide ester (“BMPS”), N-e-maleimidocaproic acid (“EMCA”), N-succinimidyl iodoacetate (“SIA”), and succinimidyl-6-[β-maleimidopropionamido]hexanoate (“SMPH”).

According to one approach, a bi-functional linker molecule such as succinimidyl-6-[βmaleimidopropionamido]hexanoate (“SMPH”) can be reacted in excess with VLPs or capsomeres. SMPH is an amine- and sulfhydryl-reactive hetero-bifunctional cross-linker. The SMPH-bound VLPs or capsomeres can be exposed to a suitable dIII polypeptide (containing a desired epitope and, preferably, a recombinantly introduced N-terminal or C-terminal cysteine residue) under conditions effective to allow for covalent binding of the dIII polypeptide to the linker molecule. After conjugation, the chimeric VLPs or capsomeres can be purified (to remove) unreacted peptide via dialysis.

Having purified the capsomeres or VLPs, these materials can be introduced into pharmaceutical compositions that are suitable for use in immunizing an individual against Flavivirus infection. Preferably, the capsomeres or VLPs are present in the pharmaceutical compositions in an amount that is effective to induce a high-titer neutralizing antibody response against the dIII epitopes and/or a TH-1 dominant CTL response. Thus, effective amounts include an amount ranging from about 1 to about 500 μg of the VLPs or capsomeres, preferably about 5 to about 200 μg, more preferably about 10 to about 100 μg, most preferably 20 to about 80 μg.

Another aspect of the present invention is directed to an immunogenic conjugate including any one of the dIII polypeptide fragments of the present invention conjugated to an immunogenic carrier molecule.

Suitable immunogenic conjugates of the present invention include, but are not limited to, an immunogenic carrier molecule covalently or non-covalently bonded to any one of the above listed dIII polypeptides. Any suitable immunogenic carrier molecule can be used. Exemplary immunogenic carrier molecules include, but are in no way limited to, bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein.

Another aspect of the present invention relates to the isolated polynucleotides that encode the above-described isolated dIII polypeptides and the isolated polynucleotides that encode any of the above-described dIII fusion proteins. In a preferred embodiment, the polynucleotide sequences encoding the isolated polypeptides or fusion proteins of the present invention are codon-optimized for expression of the polypeptide in an appropriate host cell, such as a eukaryotic or yeast host cell.

Another aspect of the present invention relates to a recombinant transgene that includes any one of the polynucleotide sequences of the present invention, including the polynucleotides encoding the dIII polypeptides or dIII-containing fusion proteins, operably coupled to a promoter-effective DNA molecule, a leader DNA sequence comprising a start-codon, and a transcription termination sequence. Selection of a suitable promoter-effective DNA molecule and other components of the recombinant transgene should be tailored to the expression system and host cell used to facilitate expression. A number of suitable promoter molecules are described infra.

Another aspect of the present invention is directed to a recombinant vector comprising any one of the above described polynucleotides or recombinant transgenes of the present invention. In accordance with this aspect of the present invention, the recombinant vector can contain any of the polynucleotides encoding the dIII polypeptides or dIII-containing fusion proteins, or the above described recombinant transgenes.

In accordance with this aspect of the invention, the polynucleotides of the present invention are inserted into an expression system or vector to which the molecule is heterologous. The heterologous nucleic acid molecule is inserted into the expression system or vector in proper sense (5′→3′) orientation relative to the promoter and any other 5′ regulatory molecules, and correct reading frame. The preparation of the nucleic acid constructs can be carried out using standard cloning methods well known in the art as described by SAMBROOK AND RUSSELL, MOLECULAR CLONING: A LABORATORY MANUAL (Cold Springs Laboratory Press, 2001), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Suitable expression vectors include those which contain replicon and control sequences that are derived from species compatible with the host cell. For example, if E. coli is used as a host cell, plasmids such as pUC19, pUC18 or pBR322 may be used. When using insect host cells, appropriate transfer vectors compatible with insect host cells include, pVL1392, pVL1393, pAcGP67 and pAcSecG2T, which incorporate a secretory signal fused to the desired protein, and pAcGHLT and pAcHLT, which contain GST and 6×His tags (BD Biosciences, Franklin Lakes, N.J.). Viral vectors suitable for use in carrying out this aspect of the invention include, adenoviral vectors, adeno-associated viral vectors, vaccinia viral vectors, nodaviral vectors, and retroviral vectors. Other suitable expression vectors are described in SAMBROOK AND RUSSELL, MOLECULAR CLONING: A LABORATORY MANUAL (Cold Springs Laboratory Press, 2001), which is hereby incorporated by reference in its entirety. Many known techniques and protocols for manipulation of nucleic acids, for example in preparation of nucleic acid constructs, mutagenesis, sequencing, introduction of DNA into cells and gene expression, and analysis of proteins, are described in detail in CURRENT PROTOCOLS IN MOLECULAR BIOLOGY (Fred M. Ausubel et al. eds., 2003), which is hereby incorporated by reference in its entirety.

Different genetic signals and processing events control many levels of gene expression (e.g., DNA transcription and messenger RNA (“mRNA”) translation) and subsequently the amount of dIII polypeptides and dIII-containing fusion proteins that are produced and expressed by the host cell. Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression. Depending upon the host system utilized, any one of a number of suitable promoters may be used. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. When using insect cells, suitable baculovirus promoters include late promoters, such as 39K protein promoter or basic protein promoter, and very late promoters, such as the p10 and polyhedron promoters. In some cases it may be desirable to use transfer vectors containing multiple baculoviral promoters.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and promote binding of mRNA to ribosomes by duplexing with the rRNA to allow correct positioning of the ribosome. For a review on maximizing gene expression, see Roberts and Leauer, “Maximizing Gene Expression on a Plasmid Using Recombination in Iiro.” Method in Enzymology, 68:473-82 (1979), which is hereby incorporated by reference in its entirety.

Host cells suitable for expressing the Dengue dIII polypeptides, fusion proteins, or recombinant transgenes include any one of the more commonly available gram negative bacteria. Suitable microorganisms include Pseudomonas aeruginosa, Escherichia coli, Salmonella gastroenterilis (typhimirium), S. lyphi, S. enteriditis, Shigella flexneri, S. sonnie, S. dyseneriae, Neisseria gonorrhoeae, N. meningitides, Haemophilus influenzae, H. pleuropneumoniae, Pasteurella haemolytica, P. multilocida, Legionella pneumophila, Treponema pallidum, T. denticola, T. orales, Borrelia burgdorferi, Borrelia spp., Leptospira interrogans, Klebsiella pneumoniae, Proteus vulgaris, P. morganii, P. mirabilis, Rickettsia prowazeki, R. typhi, R. richetisii, Porphyromonas (Bacteriodes) gingivalis, Chlamydia psittaci, C. pneumoniae, C. trachomatis, Campylobacter jejuni, C. intermedis, C. fetus, Helicobacter pylori, Francisella tularenisis, Vibrio cholerae, Vibrio parahaemolyticus, Bordetella pertussis, Burkholderie pseudomallei, Brucella abortus, B. susi, B. melitensis, B. canis, Spirillum minus, Pseudomonas mallei, Aeromonas hydrophila, A. salmonicida, and Yersinia pestis.

In addition to bacterial cells, animal cells, in particular mammalian and insect cells, yeast cells, fungal cells, plant cells, or algal cells are also suitable host cells for transfection/transformation of the recombinant expression vector carrying an isolated polynucleotide molecule of the present invention. Mammalian cell lines commonly used in the art include Chinese hamster ovary cells, HeLa cells, baby hamster kidney cells, COS cells, and many others. Suitable insect cell lines include those susceptible to recombinant baculovirus infection, including Sf9 and Sf21 cells.

Methods for transforming/transfecting host cells with expression vectors are well-known in the art and depend on the host system selected, as described in SAMBROOK AND RUSSELL, MOLECULAR CLONING: A LABORATORY MANUAL (Cold Springs Laboratory Press, 2001), which is hereby incorporated by reference in its entirety. For bacterial cells, suitable techniques include calcium chloride transformation, electroporation, and transfection using bacteriophage. For eukaryotic cells, suitable techniques include calcium phosphate transfection, DEAE-Dextran, electroporation, liposome-mediated transfection, and transduction using retrovirus or any other viral vector. For insect cells, the transfer vector containing the polynucleotide construct of the present invention is co-transfected with baculovirus DNA, such as AcNPV, to facilitate the production of a recombinant virus resulting from homologous recombination between the polynucleotide construct (encoding the dIII polypeptide) in the transfer vector and baculovirus DNA. Subsequent recombinant viral infection of Sf cells results in a high rate of recombinant protein production. Regardless of the expression system and host cell used to facilitate protein production, the expressed polypeptides and fusion proteins of the present invention can be readily purified using standard purification methods known in the art and described in PHILIP L. R. BONNER, PROTEIN PURIFICATION (Routledge 2007), which is hereby incorporated by reference in its entirety. In one embodiment, the dIII polypeptide or fusion proteins can be provided with a short amino acid sequence that aids in purification, e.g., using affinity purification techniques.

Having purified the dIII polypeptide or fusion proteins containing the same, these materials can be introduced into pharmaceutical compositions that are suitable for use in immunizing an individual against Flavivirus infection. Preferably, the dIII polypeptide or fusion proteins are present in the pharmaceutical compositions in an amount that is effective to induce a high-titer neutralizing antibody response against the dIII epitopes and/or a TH-1 dominant CTL response. Effective amounts include, without limitation, an amount ranging from about 100 ng to about 500 μg of the dIII polypeptide or fusion proteins, preferably about 1 μg to about 200 μg, more preferably about 1 to about 100 μg, most preferably 5 to about 50 μg. For multivalent vaccines, the amount of dIII polypeptides or fusion proteins can differ so as to present a balanced, neutralizing immune response against the relevant Flaviviruses.

The present invention is also directed to isolated antibodies having antigen specificity for the one or more neutralizing epitopes of the dIII polypeptide.

The isolated antibodies of the present invention may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The isolated antibody can be a full length antibody, monoclonal antibody (including full length monoclonal antibody), polyclonal antibody, multispecific antibody (e.g., bispecific antibody), human, hunmanizecd or chimeric antibody, and antibody fragments, e.g., Fab fragments, F(ab′) fragments, fragments produced by a Fab expression library, epitope-binding fragments of any of the above, and engineered forms of antibodies, e.g., scFv molecules, so long as they exhibit the desired activity, e.g., neutralizing activity against any one of Dengue serotypes 1-4.

Polyclonal antibodies can be prepared by any method known in the art. Polyclonal antibodies can be raised by immunizing an animal (e.g., a rabbit, rat, mouse, donkey, etc.) with multiple subcutaneous or intraperitoneal injections of the relevant antigen, e.g., an isolated dIII polypeptide fragment, fusion protein, or immunogenic conjugate) diluted in sterile saline and combined with an adjuvant to form a stable emulsion. The polyclonal antibody is then recovered from blood or ascites of the immunized animal. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc. Polyclonal antiserum can also be rendered monospecific using standard procedures (see e.g., Agaton et al., “Selective Enrichment of Monospecific Polyclonal Antibodies for Antibody-Based Proteomics Efforts,” J. Chromatography A. 1043(1):33-40 (2004), which is hereby incorporated by reference in its entirety).

Monoclonal antibodies can be prepared using hybridoma methods, such as those described by Kohler and Milstein, “Continuous Cultures of Fused Cells Secreting Antibody of Predefined Specificity,” Nature 256:495-7 (1975), which is hereby incorporated by reference in its entirety. Using the hybridoma method, a mouse, hamster, or other appropriate host animal, is immunized to elicit the production by lymphocytes of antibodies that will specifically bind to an immunizing dIII antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against dIII epitopes, as determined by immunoprecipitation, immunoblotting, or by an in vitro binding assay such as radioimmunoassay (RIA) or enzyme-linked immunosorbent assay (ELISA) can then be propagated either in in vitro culture using standard methods (JAMES W. GODING, MONOCLONAL ANTIBODIES: PRINCIPLES AND PRACTICE (Academic Press 1986), which is hereby incorporated by reference in its entirety) or in vivo as ascites tumors in an animal. The monoclonal antibodies can then be purified from the culture medium or ascites fluid as described for polyclonal antibodies above, and tested in a neutralization assay to confirm their neutralizing activity against one of Dengue serotypes 1-4.

Alternatively monoclonal antibodies can also be made using recombinant DNA methods as described in U.S. Pat. No. 4,816,567 to Cabilly et al, which is hereby incorporated by reference in its entirety. Polynucleotides encoding a monoclonal antibody are isolated, from mature B-cells or hybridoma cell, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries as described (McCafferty et al., “Phage Antibodies: Filamentous Phage Displaying Antibody Variable Domains,” Nature 348:552-554 (1990); Clackson et al., “Making Antibody Fragments Using Phage Display Libraries,” Nature, 352:624-628 (1991); and Marks et al., “By-passing Immunization. Human Antibodies from V-gene Libraries Displayed on Phage,” J. Mol. Biol. 222:581-597 (1991), which are hereby incorporated by reference in their entirety).

The polynucleotide(s) encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

Another aspect of the present invention is directed to a vaccine that contains any one of the isolated, recombinant dIII polypeptides, fusion proteins, or immunogenic conjugates of the present invention. The pharmaceutical composition can alternatively contain any one of the polynucleotides or the recombinant transgene of the present invention encoding any of the isolated dIII polypeptides or fusions proteins described above. These agents can be used to generate immunity in a recipient.

According to one embodiment, a tetravalent Dengue vaccine includes effective amounts of DEN1 dIII polypeptide, DEN2 dIII polypeptide, DEN3 dIII polypeptide, DEN4 dIII polypeptide, and an adjuvant, all presented in a pharmaceutically acceptable vehicle or carrier. Amounts of the dIII polypeptides identified above vary between about 1 g and about 100 μg, more preferably about 5 μg and about 50 μg so as to afford a balanced, high-titer neutralizing immune response that exceeds a PRNT₅₀ of 150 for each Flavivirus.

According to one embodiment, a monovalent Yellow Fever virus vaccine includes an effective amount YFV dIII polypeptide and an adjuvant presented in a pharmaceutically acceptable vehicle or carrier. Amounts of the dIII polypeptides vary between about 1 g and about 100 μg, more preferably about 5 μg and about 50 μg so as to afford a high-titer neutralizing immune response that exceeds a PRNT₅₀ of 150 for YFV.

According to another embodiment, a pentavalent Dengue/Yellow Fever vaccine includes effective amounts of DEN1 dIII polypeptide, DEN2 dIII polypeptide, DEN3 dIII polypeptide, DEN4 dIII polypeptide, YFV dIII polypeptide, and an adjuvant, all presented in a pharmaceutically acceptable vehicle or carrier. Amounts of the dIII polypeptides identified above vary between about 1 μg and about 100 μg, more preferably about 5 μg and about 50 μg so as to afford a balanced, high-titer neutralizing immune response that exceeds a PRNT₅₀ of 150 for each Flavivirus.

Alternatively, the present invention also relates to a pharmaceutical composition that includes an antibody of the present invention. This type of composition can be used to afford passive immunity against Dengue virus in a recipient.

The pharmaceutical compositions of the present invention also contain a pharmaceutically acceptable carrier. Acceptable pharmaceutical carriers include solutions, suspensions, emulsions, excipients, powders, or stabilizers. The carrier should be suitable for the desired mode of delivery, discussed infra.

Pharmaceutical compositions suitable for injectable use (e.g., intravenous, intra-arterial, intramuscular, etc.) may include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to sterile liquids, such as water and oils, with or without the addition of a surfactant and other pharmaceutically and physiologically acceptable carriers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

Oral dosage formulations can include standard carriers such as pharmaceutical grades of mannitol, lactose, starch, magnesium stearate, sodium saccharine, cellulose, magnesium carbonate, etc. Suitable carriers include lubricants and inert fillers such as lactose, sucrose, or cornstarch. In another embodiment, these compounds are tableted with conventional tablet bases such as lactose, sucrose, or cornstarch in combination with binders like acacia, gum gragacanth, cornstarch, or gelatin; disintegrating agents such as cornstarch, potato starch, or alginic acid; a lubricant like stearic acid or magnesium stearate; sweetening agents such as sucrose, lactose, or saccharine; and flavoring agents such as peppermint oil, oil of wintergreen, or artificial flavorings. Generally, the ingredients are supplied either separately or mixed together in unit dosage form, for example, as a dry lyophilized powder or water free concentrate in a hermetically sealed container such as an ampule or sachette indicating the quantity of active agent.

Formulations suitable for transdermal delivery can also be prepared in accordance with the teachings of Lawson et al., “Use of Nanocarriers for Transdermal Vaccine Delivery,” Clin. Pharmacol. Ther. 82(6):641-3 (2007), which is hereby incorporated by reference in its entirety.

Formulations suitable for intranasal nebulization or bronchial aerosolization delivery are also known and can be used in the present invention (see Lu & Hickey, “Pulmonary Vaccine Delivery,” Exp. Rev. Vaccines 6(2):213-226 (2007) and Alpar et al., “Biodegradable Mucoadhesive Particulates for Nasal and Pulmonary Antigen and DNA Delivery,” Adv. Drug Deliv. Rev. 57(3):411-30 (2005), which are hereby incorporated by reference in their entirety.

The pharmaceutical compositions of the present invention can also include an effective amount of an adjuvant. In pharmaceutical compositions containing a dIII polypeptide or fusion protein, an additional, preferably distinct adjuvant is included in the composition. Suitable adjuvants include, without limitation, Freund's complete or incomplete, mineral gels such as aluminum, aluminum hydroxide, surface active substances such as lysolecithin, pluronic polyols, polyanions, peptides, oil emulsions, dinitrophenol, and potentially useful human adjuvants such as Bacille Calmette-Guerin, Carynebacterium parvum, non-toxic Cholera toxin, flagellin, iscomatrix, liposome polycation DNA particles, ASO4 (an adjuvant system including a mixture of aluminum hydroxide and monophosphoryl lipid A), and HPV L1-containing VLPs or capsomeres (such as those described in PCT International Pat. Pub. WO99/61052 to Rose et al. and PCT International Pat. Pub. WO94/20137 to Rose et al., each of which is hereby incorporated by reference in its entirety). In preferred embodiments, the adjuvant is suitable for administration to humans.

The present invention also relates to a method of inducing a neutralizing immune response against Dengue serotypes 1-4 in a subject. This method involves administering to the subject dIII polypeptides or dIII-containing fusion peptides of the present invention or a pharmaceutical composition comprising the same in an amount effective to induce a neutralizing immune response against each of Dengue serotypes 1-4. The administration can be carried out as a single dose or multiple doses given over a period of time, e.g., weeks or months or even years apart. The immune response generated by such administration is preferably a high-titer neutralizing immune response (PRNT₅₀ exceeding 150) and one that is balanced against the DEN1-4 targets.

The present invention also relates to a method of inducing a neutralizing immune response against other Flaviviruses in a subject. This method involves administering to the subject dIII polypeptides or dIII-containing fusion peptides of the present invention or a pharmaceutical composition comprising the same in an amount effective to induce a neutralizing immune response against the Flavivirus, including against each of one or more serotypes of the Flavivirus. The administration can be carried out as a single dose or multiple doses given over a period of time, e.g., weeks or months or even years apart. The immune response generated by such administration is preferably a high-titer neutralizing immune response (PRNT₅₀ exceeding 150) and, if multivalent, then one that is balanced against the several Flavivirus targets.

In one embodiment, an effective immune response can be generated against each of DEN1-DEN4 and YFV using a pentavalent vaccine formulation of the invention. The immune response generated by such administration is preferably a high-titer neutralizing immune response (PRNT₅₀ exceeding 150) that is balanced against each of DEN1-DEN4 and YFV.

It is contemplated that the individual to be treated in accordance with the present invention can be any mammal, but preferably a human. Veterinary uses are also contemplated. Moreover, as noted above, the active or passive vaccine formulations are preferably tetravalent for Dengue, containing antigen directed to each of Dengue serotypes 1-4, which provides a more protective immune response; or pentavalent for Dengue and YFV. The individual to be treated can be an infant or juvenile, an elderly individual, an individual having a cardiopulmonary or immunosuppressive condition, or even an otherwise healthy adult.

Effective amounts of the composition used to induce an immune response against Dengue or other Flavivirus will depend upon the mode of administration, frequency of administration, nature of the treatment, age and condition of the individual to be treated, and the type of pharmaceutical composition used to deliver the compound. While individual doses may vary, optimal ranges of the effective amounts may be determined by one of ordinary skill in the art.

The pharmaceutical composition can be administered by any means suitable for producing the desired immune response. Preferred delivery routes include orally, by inhalation, by intranasal instillation, topically, transdermally, parenterally, subcutaneously, intravenous injection, intra-arterial injection, intramuscular injection, intraplurally, intraperitoneally, or by application to mucous membrane. The composition can be delivered repeatedly over a course of time, i.e., according to a prime/boost regiment, that achieves optimal enhancement of the immune response.

The dIII polypeptide or dIII-containing fusion proteins of the present invention, and pharmaceutical compositions comprising the same can be incorporated into a delivery vehicle to facilitate administration. Such delivery vehicles include, but are not limited to, biodegradable microspheres (MARK E. KEEGAN & W. MARK SALTZMAN, Surface Modified Biodegradable Microspheres for DNA Vaccine Delivery, in DNA VACCINES: METHODS AND PROTOCOLS 107-113 (W. Mark Saltzman et al., eds., 2006), which is hereby incorporated by reference in its entirety), microparticles (Singh et al., “Nanoparticles and Microparticles as Vaccine Delivery Systems,” Expert Rev. Vaccine 6(5):797-808 (2007), which is hereby incorporated by reference in its entirety), nanoparticles (Wendorf et al., “A Practical Approach to the Use of Nanoparticles for Vaccine Delivery,” J. Pharmaceutical Sciences 95(12):2738-50 (2006) which is hereby incorporated by reference in its entirety), liposomes (U.S. Patent Application Publication No. 20070082043 to Dov et al. and Hayashi et al., “A Novel Vaccine Delivery System Using Immunopotentiating Fusogenic Liposomes,” Biochem. Biophys. Res. Comm. 261(3): 824-28 (1999), which are hereby incorporated by reference in their entirety), collagen minipellets (Lofthouse et al., “The Application of Biodegradable Collagen Minipellets as Vaccine Delivery Vehicles in Mice and Sheep,” Vaccine 19(30):4318-27 (2001), which is hereby incorporated by reference in it entirety), and cochleates (Gould-Fogerite et al., “Targeting Immune Response Induction with Cochleate and Liposome-Based Vaccines,” Adv. Drug Deliv. Rev. 32(3):273-87 (1998), which is hereby incorporated by reference in its entirety).

The compositions of the present invention can further be formulated for the desired mode of administration. For example, the composition can be formulated into a single-unit oral dosage, an injectable dose contained in a syringe, a transdermally deliverable dosage contained in a transdermal patch, or an inhalable dose contained in an inhaler.

For prophylactic treatment against Dengue infection, it is intended that the composition(s) of the present invention can be administered prior to exposure of an individual to Dengue virus serotypes 1-4 and that the resulting immune response can inhibit or reduce the severity of the Dengue infection such that the Dengue virus can be eliminated from the individual. The pharmaceutical compositions of the present invention can also be administered to an individual for therapeutic treatment. In accordance with embodiment, it is intended that the antibody composition(s) of the present invention can be administered to an individual who is already exposed to the Dengue virus. This can reduce the duration or severity of the existing Dengue infection, as well as minimize any harmful consequences of untreated Dengue infections. The composition(s) can also be administered in combination other therapeutic anti-Dengue regimen.

For prophylactic treatment against other Flavivirus infection, it is intended that the composition(s) of the present invention can be administered prior to exposure of an individual to Flavivirus and that the resulting immune response can inhibit or reduce the severity of the Flavivirus infection such that the virus can be eliminated from the individual. The pharmaceutical compositions of the present invention can also be administered to an individual for therapeutic treatment. In accordance with embodiment, it is intended that the antibody composition(s) of the present invention can be administered to an individual who is already exposed to the Flavivirus. This can reduce the duration or severity of the existing Flavivirus infection, as well as minimize any harmful consequences of untreated viral infections. The composition(s) can also be administered in combination other therapeutic anti-Flavivirus regimen.

### EXAMPLES

The following examples are provided to illustrate embodiments of the present invention but are by no means intended to limit its scope.

### Materials and Methods for Examples 1-6

Animals

Female BALB/c mice (8-10 weeks of age) were obtained from Taconic Laboratories (Germantown, N.Y.). All procedures were performed in accordance with University of Rochester Committee on Animal Resources approved protocols for animal use.

Cells and Viruses

C6/36 Aedes albopictus mosquito cells were grown at 28° C. in modified Eagle's medium (MEM) supplemented with sodium pyruvate and nonessential amino acids. African green monkey kidney-derived Vero cells were propagated in MEM supplemented with fetal bovine serum (FBS). K562 and U937 cells were cultured in RPMI-1640 supplemented with heat-inactivated FBS. Cells were cultured in a 5% CO2 environment. DENVs representative of each of the four DENV serotypes (DENV1-16007; DENV2-16681; DENV3-16562; DENV4-1036) were gifts of Dr. Richard Kinney (CDC, Ft. Collins, Colo.) and were propagated in mosquito cells. Virus titers were determined by immunostain plaque assay on Vero cell monolayers (Shanaka et al., “An Automated Dengue Virus Microneutralization Plaque Assay Performed in Human Fc{Gamma} Receptor-Expressing CV-1 Cells,” Am. J. Trop. Med. Hyg. 80(1):61-5 (2009), which is hereby incorporated by reference in its entirety).

Antibodies

DENV dIII-specific monoclonal antibodies included: mAb DV1-E50 (DENV1) (a gift from Michael S. Diamond, Wash U) (Rodrigo et al., “Dengue Virus Neutralization is Modulated By IgG Antibody Subclass and Fcgamma Receptor Subtype,” Virology 394(2):175-82 (2009), which is hereby incorporated by reference in its entirety), mAb 1F1 (DENV2) (Sukupolvi-Petty et al., “Type- and Subcomplex-Specific Neutralizing Antibodies Against Domain III of Dengue Virus Type 2 Envelope Protein Recognize Adjacent Epitopes,” J. Virol. 81(23):12816-26 (2007), which is hereby incorporated by reference in its entirety), and 8A1 (DENV3) (a gift from Mary K. Gentry, WRAIR). DENV serotype-specific reference mouse immune ascites fluid (MIAF, CDC, Ft Collins, Colo.) corresponding to each of the four DENV serotypes were prepared by hyperimmunization with live DENV1-Hawaii, DENV2-NGC, DENV3-H87, or DENV4-H241. A human serum pool that neutralized all DENV serotypes has been previously described (Rodrigo et al., “Differential Enhancement of Dengue Virus Immune Complex Infectivity Mediated By Signaling-Competent and Signaling-Incompetent Human Fcgamma RIA (CD64) or FcgammaRIIA (CD32),” J. Virol. 80(20):10128-38 (2006), which is hereby incorporated by reference in its entirety). Mouse monoclonal antibody MMS-156P, directed to the 6HIS epitope tag, was from Covance, Berkley, Calif. Humanized chimpanzee flavivirus cross-reactive antibody 1A5, (Goncalvez et al., “Monoclonal Antibody-Mediated Enhancement of Dengue Virus Infection in Vitro and in Vivo and Strategies for Prevention,” Proc. Natl. Acad. Sci. U.S.A. 104(22):9422-7 (2007), which is hereby incorporated by reference in its entirety) was a gift of Dr. C-J Lai (NIH). APC-labeled mouse antibody 7E1 against envelope E protein of all four DENV serotypes has previously been described (Kou et al., “Monocytes, But Not T or B Cells, are the Principal Target Cells for Dengue Virus (DV) Infection Among Human Peripheral Blood Mononuclear Cells,” J. Med. Virol. 80(1):134-46 (2008), which is hereby incorporated by reference in its entirety).

Plasmid DNA Constructs

Genomic RNA was extracted from the supernatants of C6/36 cells infected with each of the four reference strain viruses (FIGS. 1A-C) and used as a template for RT-PCR with DENV dIII-specific primers. The dIII region of each DENV serotype was cloned individually into the pAcGP67A (Pharmingen, San Diego, Calif.) baculovirus transfer vector. Each DENV-dIII coding region was fused to an amino-terminal glycoprotein gp67 leader sequence derived from the Autographa californica nuclear polyhedros virus (AcNPV) to facilitate secretion of recombinant protein into infected cell supernatants, and to a carboxy-terminal polyhistidine tag for metal affinity purification. Nucleotide sequences were verified by BLAST analysis.

Recombinant DENV dIII Protein Expression, Purification and Characterization

Methods used for the generation of recombinant baculoviruses that mediate expression of proteins in insect cells have been described previously in detail (Rose et al., “Expression of Human Papillomavirus Type 11 L1 Protein in Insect Cells: in Vivo and in Vitro Assembly of Viruslike Particles,” J. Virol. 67(4):1936-44 (1993); Rose et al., “Expression of the Full-Length Products of the Human Papillomavirus Type 6b (HIPV-6b) and HPV-11 L2 Open Reading Frames by Recombinant Baculovirus, and Antigenic Comparisons With HPV-11 Whole Virus Particles,” J. Gen. Virol. 71 (Pt 11):2725-9 (1990); Rose et al., “Serological Differentiation of Human Papillomavirus Types 11, 16 and 18 Using Recombinant Virus-Like Particles,” J. Gen. Virol. 75 (Pt 9):2445-9 (1994), all of which are hereby incorporated by reference in their entirety). Briefly, Trichoplusia ni insect cells (High Five™ cells, Invitrogen, Carlsbad, Calif.) were propagated in 300-mL shake cultures (125 rpm, 27° C.) in Express Five serum-free medium (Invitrogen) and were infected at a multiplicity of infection (MOI)=3. Cell cultures were incubated with shaking for 72 hours at 27° C. Supernatants containing secreted recombinant proteins were clarified by centrifugation (800×g) and incubated with Talon metal affinity resin (Talon Metal Affinity Purification, BD Biosciences, Palo Alto, Calif.) for metal affinity chromatography. Proteins were eluted from beads using 10 mM imidazole and dialyzed against PBS. Protein concentration was determined by bicinchoninic acid assay (Pierce, Rockford, Ill.). Recombinant proteins (200 ng) were resolved by 15% SDS-PAGE and visualized with Coomassie brilliant blue (Sigma, St. Louis, Mo.). Proteins were transferred to nitrocellulose membranes and immunoblots were performed with monoclonal or polyclonal antibodies.

Mouse Immunization

Recombinant DENV dIII proteins were emulsified individually (10 μg per dose) or in tetravalent combination (5 μg to 50 μg per dose) in complete Freund's adjuvant (CFA, Sigma, St. Lois, Mo.) for priming (day 0), and in incomplete Freund's adjuvant (IFA) for booster immunizations (days 14 and 28). DENV dIII protein doses were delivered in a uniform 80 μl volume by hind leg intramuscular (i.m.) injection. Blood was collected on day −2, 12, and 26 by retro-orbital bleed, and by terminal cardiac puncture on day 42.

Antibody Specificity and Isotype Measurement

Anti-DENV dIII mouse antibodies were measured by ELISA performed in 96-well plates (NUNC immobilizer, Nunc, Rochester, N.Y.) coated with 50 ng of the respective DENV dIII protein by overnight adsorption, or by intact DENV2 virions captured in the solid phase by primate mAb 1A5 using a previously described ELISA method (Rodrigo et al., “Dengue Virus Neutralization is Modulated By IgG Antibody Subclass and Fcgamma Receptor Subtype,” Virology 394(2): 175-82 (2009), which is hereby incorporated by reference in its entirety). Washed plates were developed with alkaline phosphatase conjugated sheep-anti-mouse secondary antibody (GE Healthcare, Piscataway, N.J.). Since the DENV dIII proteins used in the current experiment were 6HIS-tagged, mouse DENV dIII immune sera used for dIII immunoblots were pre-adsorbed with an irrelevant 6HIS-tagged protein (recombinant bacteriophage 6HIS-gpD) immobilized on nitrocellulose membranes. Anti-DENV specific IgG subclass distribution was determined by indirect ELISA (Clono-typing kit, Southern Biotechnology Associates, Inc., Birmingham, Ala.) using DENV2 dIII protein or virion in the solid phase, according to the manufacturer's protocol.

Neutralization and Enhancement Tests

Antibody-mediated DENV neutralization in Vero cells was determined by a previously described microneutralization plaque assay in Vero cells (Shanaka et al., “An Automated Dengue Virus Microneutralization Plaque Assay Performed in Human Fc{Gamma} Receptor-Expressing CV-1 Cells,” Am. J. Trop. Med. Hyg. 80(1):61-5 (2009), which is hereby incorporated by reference in its entirety). Percent plaque reduction and PRNT₅₀ titers were calculated by probit analysis (Russell et al., “A Plaque Reduction Test for Dengue Virus Neutralizing Antibodies,” J. Immunol. 99(2):285-90 (1967), which is hereby incorporated by reference in its entirety) using GraphPad Prism software v5.0. Antibody-mediated enhancement of DENV2 infectivity was measured by flow cytometry in K562 and U937 cells as previously described (Goncalvez et al., “Monoclonal Antibody-Mediated Enhancement of Dengue Virus Infection in Vitro and in Vivo and Strategies for Prevention,” Proc. Natl. Acad. Sci. U.S.A. 104(22):9422-7 (2007), which is hereby incorporated by reference in its entirety). Briefly, immune complexes formed by incubating serially diluted antibody and virus were incubated with K562 (MOI0.05) or U937 (MOI=5) cells for 90 minutes at 37° C. Cells were then washed with PBS, re-suspended in fresh medium, and incubated for 48 hours at 37° C. Infections with and without virus were performed in parallel as controls. Cells were stained for intracellular DENV virion E protein with Alexa 647-labeled mAb 7E1 (Rodrigo et al., “Differential Enhancement of Dengue Virus Immune Complex Infectivity Mediated By Signaling-Competent and Signaling-Incompetent Human Fcgamma RIA (CD64) or FcgammaRIIA (CD32),” J. Virol. 80(20):10128-38 (2006), which is hereby incorporated by reference in its entirety) and counted using a BD LSRII instrument and analyzed using FlowJo software.

Statistics

Kruskal-Wallis test and Dunn's Multiple Comparison post-test were performed using GraphPad PRISM software, v5.0; P<0.05 was considered statistically significant.

### Example 1

### Expression and Purification of Recombinant DENV dIII Proteins

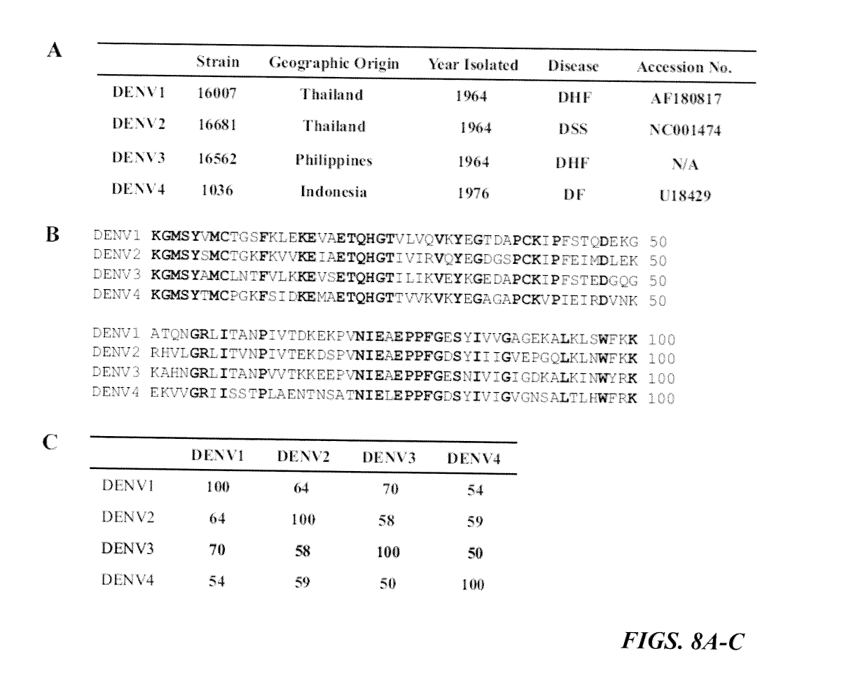

Summarized in FIGS. 8A-C are genetic characteristics of the four DENV serotypes chosen to prepare DENV dIII protein immunogens for this study; the origin and properties of each DENV have been previously described (Halstead et al., “Biologic Properties of Dengue Viruses Following Serial Passage in Primary Dog Kidney Cells: Studies at the University of Hawaii,” Am. J. Trop. Med. Hyg. 69(6 Suppl):5-11 (2003), which is hereby incorporated by reference in its entirety). DENV1, DENV2, and DENV4 sequences were verified by comparison with published determinations; DENV3 16562 dIII nucleotide sequence is unpublished, but was identical to that of reference DENV3 H-87 (accession no. M93130). DENV4 dIII is notable for manifesting the lowest sequence homology with other DENV serotypes.

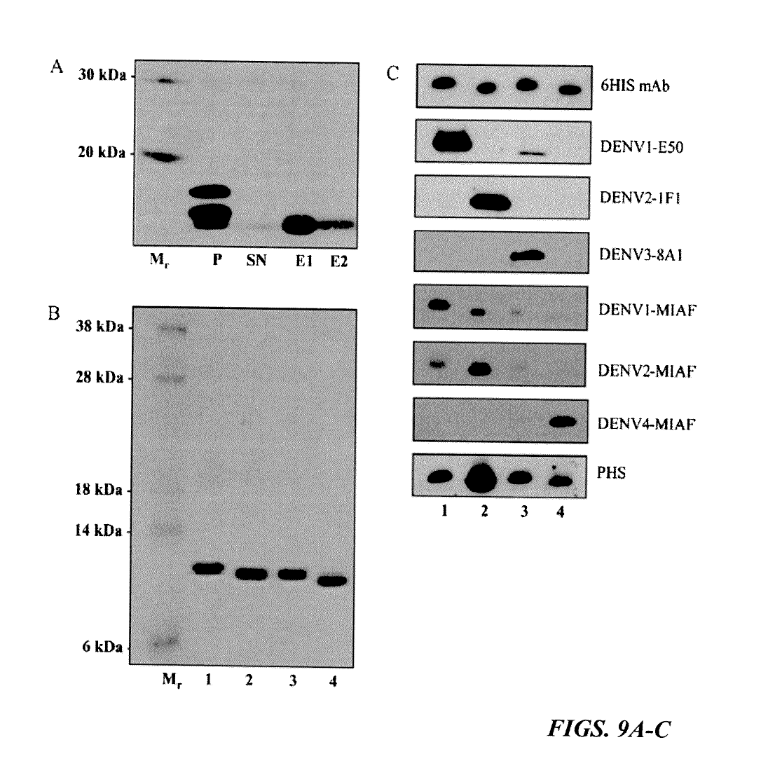

A baculovirus vector transfer system was adopted that exploited a cleavable leader sequence to promote efficient secretion of 6HIS-tagged soluble recombinant DENV dIII proteins. As expected, the metal-affinity purified DENV dIII proteins were present in both the cell pellet (P) and supernatant (SN) fractions of baculovirus-infected insect cells (FIG. 9A). Protein yields were in the range 2-10 mg/L supernatant comparing favorably with other methods used to prepare DENV dIII proteins (see Table 1, infra). Proteolytic cleavage of the leader sequence during secretion resulted in a ˜12 kDa secreted recombinant protein (larger species are unprocessed). DENV dIII proteins were purified to near homogeneity using cobalt metal affinity chromatography (FIG. 9A, 9B). Affinity-purified 6HIS-tagged DENV dIII proteins were resolved as a single band (FIG. 9B) confirming the homogeneity of each serotype-specific DENV dIII protein preparation.

TABLE 1
Recombinant Dengue Domain III Protein Antigens

| | | Soluble | Refolding | Yield | DENV1 | DENV2 | DENV3 | DENV4 | |
|---|---|---|---|---|---|---|---|---|---|
| Host | Carrier | Expression | Steps | (mg/L) | PRNT₅₀ | PRNT₅₀ | PRNT₅₀ | PRNT₅₀ | References |
| E. coli | MBP | Yes | No | NR | 100^{a} | 450^{a} | 480^{a} | 35^{a} | 1 |
| | Trx | Yes | No | NR | | | | | 2 |
| | TrpE | No | No | NR | | | | | 3 |
| | P64k | No | Yes | NR | | 640 | | | 4 |
| | | No | Yes | 30 | | | | | 5 |
| | | No | Yes | 25 | | 64 | | | 6 |
| | | No | Yes | NR | | | | 128 | 7 |
| | | Yes | No | 0.57 | | | | | 8 |
| | | Yes | No | NR | 16^{a,b} | 128^{a,b} | 32^{a,b} | 8^{a,b} | 9 |
| Yeast | | No | Yes | 50 | | 226 | | | 10 |
| | | No | Yes | NR | 160^{a,c} | 18^{a,c} | 234^{a,c} | 479^{a,c} | 11 |
| Insect | | Yes | No | 2-10 | 1196^{a} | 3174^{a} | 378^{a} | 254^{a} | Examples |
| | | | | | | | | | 1-6 herein |

^{a}tetravalent DENV dIII vaccine formulation;
^{b}consensus DENV dIII;
^{c}tetravalent tandem repeat DENV dIII antigen
The references cited above and listed below are hereby incorporated by reference in their entirety:
1 Simmons et al., Am. J. Trop. Med. Hyg. 58(5): 655-62 (1998); Simmons et al., Am. J. Trop. Med. Hyg. 65(2): 159-61 (2001).
2 Chin et al., Microbes Infect. 9(1): 1-6 (2007).
3 Fonseca et al., Am. J. Trop. Med. Hyg. 44(5): 500-8 (1991).
4 Lazo et al., Biotechnol. Appl. Biochem. 52(Pt 4): 265-71 (2009); Hermida et al., J. Virol. Methods 115(1): 41-9 (2004); Zulueta et al., Biochem. Biophys. Res. Commun. 308(3): 619-26 (2003).
5 Jaiswal et al., Protein Expr. Purif. 33(1): 80-91 (2004).
6 Zhang et al., J. Virol. Methods 143(2): 125-31 (2007).
7 Babu et al., Vaccine 26(36): 4655-63 (2008); Pattnaik et al., J. Chromatogr. B Analyt. Technol. Biomed. Life Sci. 846(1-2): 184-94 (2007).
8 Saejung et al., J. Biosci. Bioeng. 102(4): 333-9 (2006).
9 Leng et al., Microbes. Infect. 11(2): 288-95 (2009).
10 Batra et al., J. Virol. Methods. 167(1): 10-6 (2010).
11 Etemad et al., Am. J. Trop. Med. Hyg. 79(3): 353-63 (2008).

### Example 2

### Antigenic Characterization of Recombinant DENV dIII Proteins

To verify antigenic display of DENV dIII native epitopes, immunoblot analysis was performed with a panel of well-characterized DENV antibodies comprised of serotype-specific and subcomplex-specific MAbs, DENV serotype-specific mouse immune ascites, and pooled convalescent sera from DHF/DSS patients (FIG. 9C). The 6HIS mAb reacted with each DENV dIII protein confirming its correct processing and secretion. DENV subcomplex-reactive MAb DV1-E50 prepared against DENV1, also exhibited weak neutralizing activity against DENV3. Concordantly, DV1-E50 reacted strongly against DENV1 dIII and with lower intensity against DENV3 dIII. Monotypic reactivity was observed with DENV dIII lateral ridge-directed MAbs 1F1 and 8A1 which exclusively neutralize DENV2 and DENV3, respectively. No DENV4 specific mAb was available for testing, but the corresponding DENV4 mouse immune ascites exhibited monotypic reactivity, whereas some minor serotype cross-reactivity was observed with immune ascites raised against DENV1 and DENV2. Unexplainably, mouse DENV3 immune ascites that exhibited potent monotypic neutralizing activity failed to react with recombinant DENV3 dIII or other DENV dIII serotypes. Notably, convalescent sera from DHF/DSS patients reacted with each of the four DENV dIII serotypes.

Collectively, results of the present example verified DENV dIII purity and were in accord with the predicted DENV antigenic reactivity of the respective DENV dIII preparations.

### Example 3

### Monovalent DENV2 dIII Immunization Elicits Homologous Virus Neutralizing Antibodies

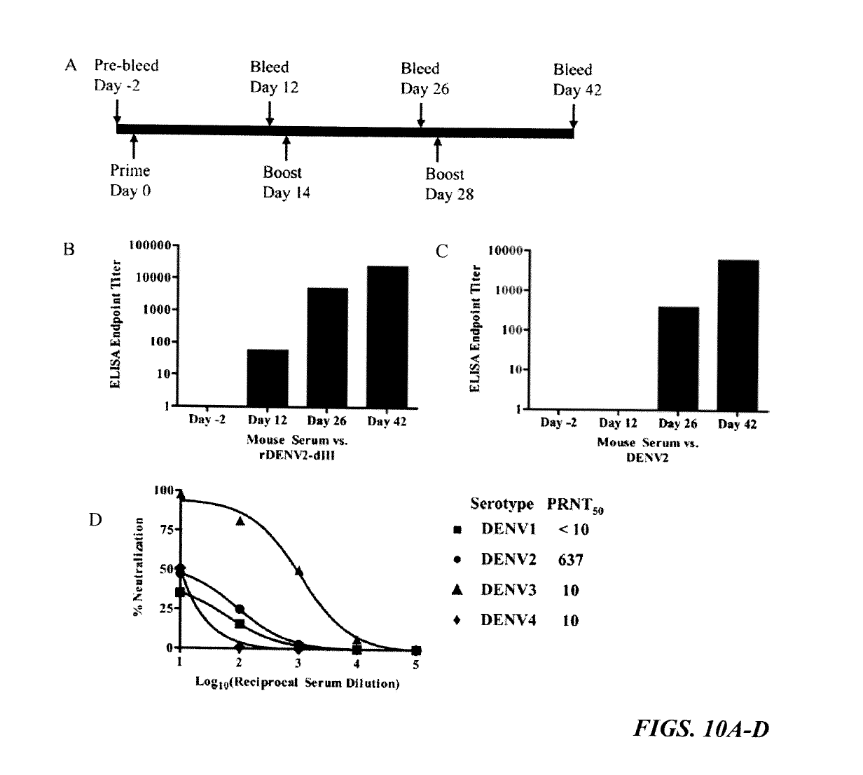

Guided by DENV dIII antibody binding results, a study of DENV dIII immunogenicity was initiated by first evaluating the capacity of DENV2 dIII protein to stimulate DENV neutralizing antibodies. Summarized in FIG. 10A is the immunization and bleed schedule of mice inoculated with DENV2-dIII (10 μg) in complete or incomplete Freund's adjuvant. This prime and boost schedule was used throughout the present study. Since antibodies generated against DENV dIII preparations of the present invention would be expected to include those directed to the 6HIS tag, the IgG response to DENV2 dIII protein or DENV2 virion in the solid phase was measured in parallel ELISAs (FIGS. 10B-C). Anti-DENV2 dIII protein and virion titers rose proportionately with sequential delivery of booster doses indicating that anti-dIII antibodies also recognized this antigen in its native virion configuration. In accord with these DENV dIII-specific binding results, neutralization assays with pooled sera collected on day 42 post-immunization, demonstrated potent homotypic neutralizing activity against DENV2 (PRNT₅₀=637) and only trivial (PRNT₅₀=0; DENV3, DENV4) or no heterologous DENV neutralizing activity (PRNT₅₀<10, DENV1) (FIG. 10D).

### Example 4

### Neutralizing Antibody Response to Tetravalent DENV dIII Formulations is Influenced by Antigen Amount

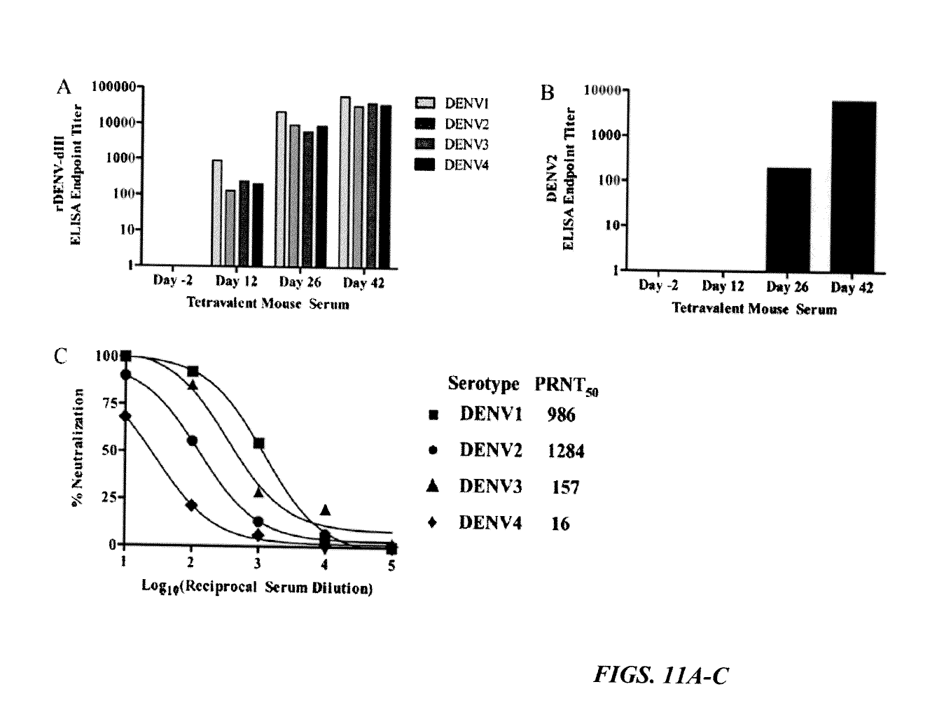

Generation of a balanced antibody neutralizing response to each DENV serotype of a tetravalent dengue vaccine is desired for its safety and efficacy. The finding of a potent, essentially monotypic, neutralizing antibody response to DENV2 dIII pointed to the possibility of a tetravalent formulation capable of stimulating DENV serotype-specific antibodies that collectively neutralize all four serotypes with little contribution by weak, cross-neutralizing antibodies. To this end, a tetravalent vaccine formulation comprised of equal amounts of each DENV dIII protein was evaluated first. Five mice were immunized with 10 μg each of a DENV dIII protein mixture and bled using the same schedule as that for monovalent DENV2 dIII immunization (FIGS. 10A-D). ELISA end-point titers against each of the four DENV dIII components increased with no discernable differences among them over the course of the immunization schedule (FIG. 11A). Increasing amounts of antibodies against DENV2 virion dIII were detected in parallel (FIG. 11B). However, despite generation of apparently similar DENV dIII-specific IgG antibody levels determined by ELISA (FIG. 11A), the neutralizing antibody response appeared notably unbalanced with PRNT₅₀ titers (GMT) against DENV1, 2, 3, and 4 being 1:986, 1:1284, 1:157, and 1:16, respectively. Variation in titers among individual mice was wide, but only the DENV4 PRNT₅₀ was statistically different (p<0.05, Kruskal-Wallis test).

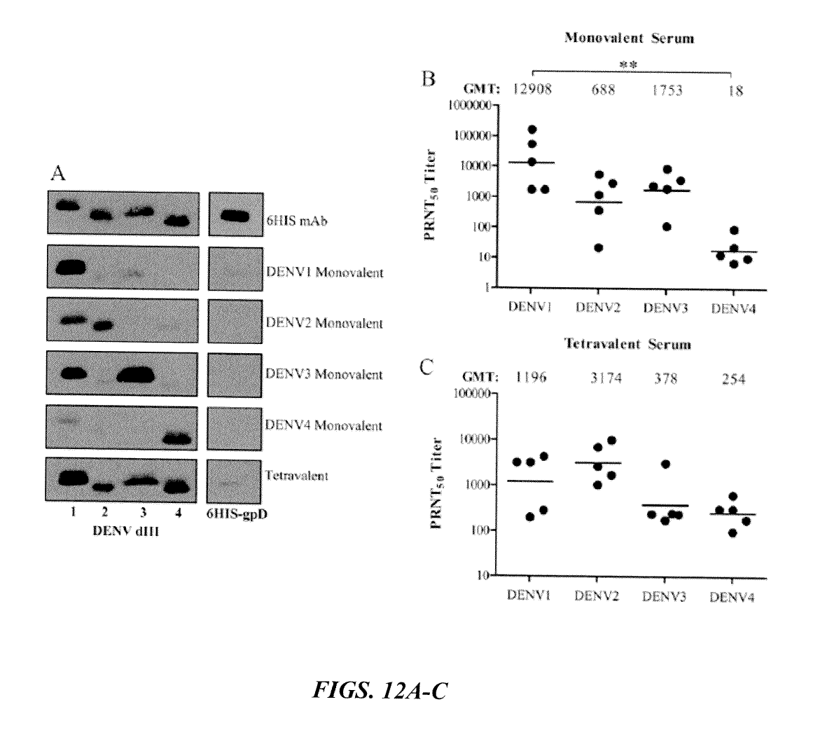

The unexpected divergence in neutralizing antibody responses among DENV serotypes following immunization with equivalent amounts of each DENV dIII preparation prompted the evaluation of the possible effect of DENV dIII protein dose modification on neutralizing antibody response. Guided by the hierarchy of responses to a uniform dose (10 μg) tetravalent formulation that stimulated an equally robust neutralizing antibody response to DENV1 and DENV2, but weaker neutralizing activity against DENV4 and seemingly, DENV3 (FIG. 11C), the dIII protein dose of these DENV serotypes was increased (and that of DENV1), while reducing that of DENV2, with the new formulation being: 25 μg DENV1 dIII; 5 μg DENV2 dIII; 25 μg DENV3 dIII; and, 50 μg DENV4 dIII. The specificity of antibodies generated by these serotype-specific DENV dIII preparations delivered individually or in tetravalent combination was assessed first. Shown in FIG. 12A are immunoblots with 6HIS pre-adsorbed pooled sera from mice immunized with monovalent or tetravalent DENV dIII preparations; relatively trivial 6HIS reactivity remained. Each monovalent preparation stimulated homotypic antibodies against the respective DENV dIII serotype as determined by immunoblot (FIG. 12A), but cross-reactive antibodies were also generated, particularly against DENV1 which exhibits relatively strong sequence homology among DENV serotypes (FIG. 8C).

As with the previous immunizations, considerable variation in DENV serotype-specific neutralization end-point titers was observed among sera from individual mice within a particular monovalent, or tetravalent DENV dIII-immunized group resulting in relatively wide standard deviations among GMTs (FIGS. 12B, 12C). Her, too, the neutralizing antibody response to monovalent DENV4 dIII immunization was significantly lower than that against the other DENV dIII serotypes (GMT= 1/18; p<0.05), and was virtually identical to that observed with the equal dose tetravalent formulation (GMT= 1/16). The neutralizing antibody response to DENV1 dIII was roughly 700-fold greater (GMT=12,908) than that to DENV4 dIII. Neutralizing antibody responses to DENV2 and DENV3 were similar (GMT=688 and 1753, respectively).

DENV neutralization end-point titers among mice immunized with the DENV dIII proteins delivered in mixed dose tetravalent formulation (FIG. 12B) were somewhat different from those measured in mice given the respective monovalent DENV dIII preparations, individually. Here, the tetravalent formulation produced a more balanced neutralizing antibody profile, with anti-DENV3 and anti-DENV4 titers comparably lower (5- to 14-fold) than those against DENV1 and DENV2, differences that were statistically insignificant. Collectively, these results demonstrate that the DENV serotype-specific neutralizing antibody response to a tetravalent DENV dIII vaccine is influenced by the relative amounts of its DENV dIII protein components.

### Example 5

### IgG Subclass Distribution Among Mouse Antibodies Stimulated by a Tetravalent DENV dIII Protein Vaccine

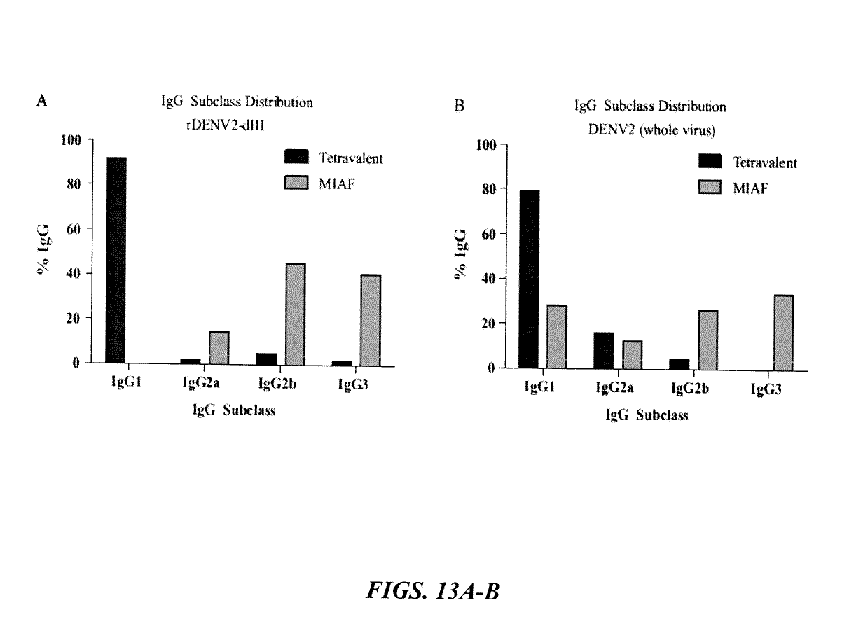

It has previously been shown that DENV neutralization is modulated in an IgG subclass manner, likely through effects of the Fc region on virion and FcγR binding (Rodrigo et al., “Dengue Virus Neutralization is Modulated By IgG Antibody Subclass and Fcgamma Receptor Subtype,” Virology 394(2): 175-82 (2009), which is hereby incorporated by reference in its entirety). Furthermore, the IgG Fc piece also governs complement fixation, and the enhancing capacity of IgG subclasses of DENV antibodies that fix complement is abrogated by C1q in FcγR-positive cells (Mehlhop et al., “Complement Protein C1q Inhibits Antibody-Dependent Enhancement of Flavivirus Infection in an IgG Subclass-Specific Manner,” Cell Host Microbe 2(6):417-26 (2007), which is hereby incorporated by reference in its entirety). These observations prompted assessment of the IgG subclass distribution among antibodies stimulated by the DENV dIII tetravalent vaccine. An indirect ELISA was used to compare the DENV-specific IgG antibody subclass profile in pooled serum from mice immunized with tetravalent formulated DENV dIII or in pooled monotypic reference sera from mice immunized with live DENV (FIGS. 13A-B). Pooled mouse immune sera (day 42 post-vaccination) from dose-adjusted tetravalent immunization comprised predominantly IgG1 DENV antibodies as determined by binding to DENV2 dIII protein (FIG. 13A) or intact DENV2 virion (FIG. 13B) in the solid phase. Serum from live virus infected mice assayed in parallel, exhibited a much more diverse IgG subclass response. These results are in accord with earlier observations of predominantly IgG1 responses to subunit viral vaccination in contrast to predominantly complement-fixing IgG2a and IgG2b responses to a replicating live virus challenge (Coutelier et al., “Virally Induced Modulation of Murine IgG Antibody Subclasses,” Exp. Med. 168(6):2373-8 (1988); Smucny et al., “Murine Immunoglobulin G subclass Responses Following Immunization With Live Dengue Virus or a Recombinant Dengue Envelope Protein,” Am. J. Trop. Med. Hyg. 53(4):432-7 (1995); Simmons et al., “Characterization of Antibody Responses to Combinations of a Dengue-2 DNA and Dengue-2 Recombinant Subunit Vaccine,” Am. J. Trop. Med. Hyg. 65(5):420-6 (2001), all of which are hereby incorporated by reference in their entirety).

### Example 6

### Tetravalent DENV dIII Immune Sera Mediate ADE

Immunization of mice with infectious DENV or its dIII protein induced both potent DENV serotype-specific neutralizing antibodies and, generally, less potent DENV sub-complex antibodies (Sukupolvi-Petty et al., “Type- and Subcomplex-Specific Neutralizing Antibodies Against Domain III of Dengue Virus Type 2 Envelope Protein Recognize Adjacent Epitopes,”J. Virol. 81(23):12816-26 (2007); Gromowski et al., “Characterization of Dengue Virus Complex-Specific Neutralizing Epitopes on Envelope Protein Domain III of Dengue 2 Virus,” J. Virol. 82(17):8828-37 (2008), which are hereby incorporated by reference in their entirety). Weakly neutralizing sub-complex (i.e., “cross-reactive”) DENV antibodies, especially those with the property of low-affinity FcγR binding (e.g., mouse IgG1) might particularly be expected to mediate ADE (Rodrigo et al., “Dengue Virus Neutralization is Modulated By IgG Antibody Subclass and Fcgamma Receptor Subtype,” Virology 394(2):175-82 (2009), which is hereby incorporated by reference in its entirety). IgG1 antibodies that included heterotypic IgG antibodies of weak neutralizing activity predominated after tetravalent DENV dIII immunization prompting the measurement of their capacity to mediate ADE. To accomplish this, two FcγR-expressing cell lines were used that have been widely used for DENV ADE measurements. The first, K562 of erythroid lineage, is highly permissive to DENV infection in the absence of DENV antibodies; it displays FcγRIIA (CD32) only. The second, U937 of monocyte/macrophage lineage, is relatively insusceptible to DENV infection in the absence of DENV antibodies: it displays both FcγRIA (CD64) and FcγRIIA (CD32). Both CD32 and CD64 bind mouse IgG1 antibodies with similar low affinity.

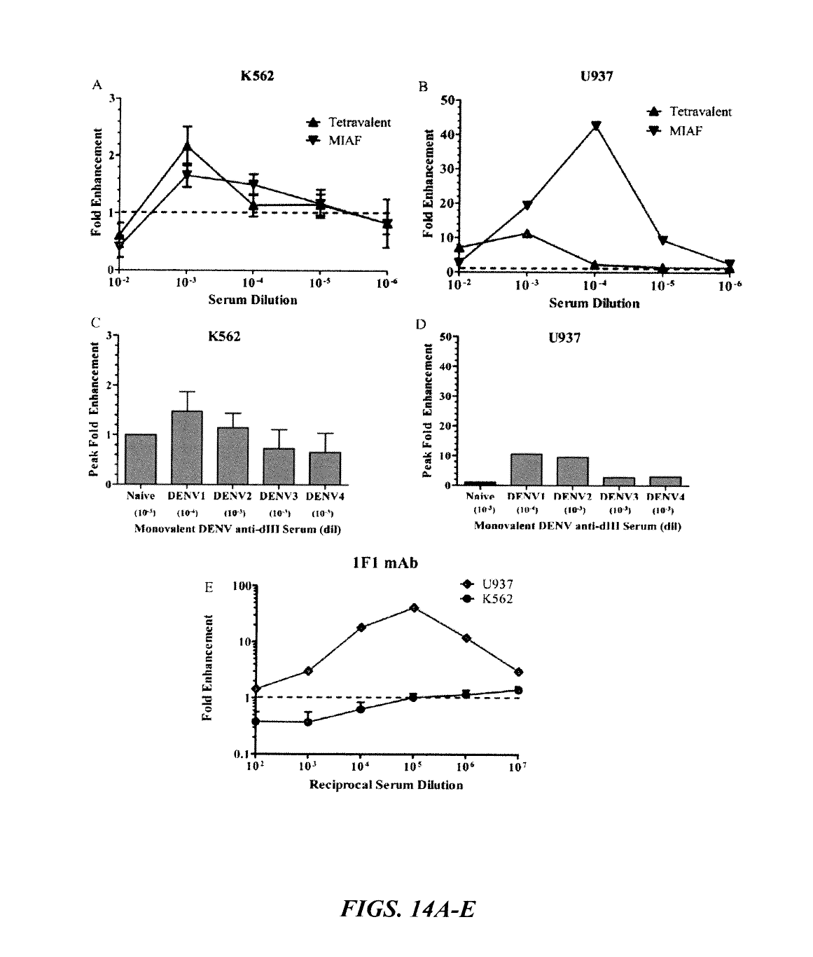

In K562 cells (FIG. 14A), a similar roughly 2-fold peak enhancement was observed with both tetravalent DENV dIII serum and comparator polyvalent mouse immune serum at the same serum dilution (1/1000). Neutralization was observed with both antisera at the lowest dilution tested (1/100). In U937 cells (FIG. 14B), peak enhancement by tetravalent DENV dIII serum was notably lower than that of the comparator polyvalent mouse serum (˜10-fold vs ˜40-fold), which was also observed over a much wider antibody dilution range. DENV2 ADE was also mediated in both cell types by monotypic DENV1 and DENV2 dIII mouse immune sera, but not by DENV3 or DENV4 monotypic immune sera. Shown in FIGS. 14C, 14D are relative ADE levels among monotypic immune sera used at single serum dilutions that mediated peak enhancement in preliminary ranging experiments with each cell type. Notably, mAb 1F1 a DENV2 serotype-specific neutralizing IgG2a antibody directed to the dIII lateral ridge (Sukupolvi-Petty et al., “Type- and Subcomplex-Specific Neutralizing Antibodies Against Domain III of Dengue Virus Type 2 Envelope Protein Recognize Adjacent Epitopes,”J. Virol. 81(23):12816-26 (2007), which is hereby incorporated by reference in its entirety), exhibited predominantly neutralizing activity in K562 cells (FIG. 14E) whereas it promoted ADE in U937 cells over a wide range of dilutions reaching a peak enhancement level that was ˜5-fold greater than that observed with the tetravalent antibody preparation in these cells (FIG. 14B).

### Discussion of Examples 1-6

Summarized in Table 1 (see supra) are published recombinant DENV dIII-based candidate subunit vaccines, which were prepared in bacterial or yeast based systems. Some incorporated a fusion partner to increase DENV dIII protein solubility, whereas others were solubilized by extensive refolding steps. Most stimulated neutralizing antibodies, although none generated a significant immune response (PRNT₅₀ titers>1:150 for all four serotypes) and their fine antigenic specificity or capacity to mediate ADE was not measured. The preceding Examples demonstrate that a tetravalent Dengue vaccine can be formulated to generate a balanced DENV serotype-specific neutralizing antibody response in mice using recombinant DENV dIII proteins secreted by insect cells. The preceding Examples also demonstrate that it is also possible to elicit a reduction in antibodies that mediate ADE in FcγR-positive cells.

Several main points emerge from the data of the preceding Examples. First, the soluble insect cell-derived recombinant DENV dIII proteins are secreted in relatively copious amounts in a manner suitable for scale-up production and no need for further modifying steps. Importantly, they are recognized by a diverse panel of DENV neutralizing antibodies and immune sera including sera from DHF/DSS patients, in keeping with a DENV dIII antibody response in human DENV infection. These results indicate that key DENV neutralization determinants are preserved in the DENV dIII protein preparations, which has not uniformly been the case with previously reported recombinant DENV dIII proteins, particularly after fusion protein cleavage or when denaturation was required to solubilize such DENV dIII proteins (Simmons et al., “Evaluation of the Protective Efficacy of a Recombinant Dengue Envelope B Domain Fusion Protein Against Dengue 2 Virus Infection in Mice,” Am. Trop. Med. Hyg. 58(5):655-62 (1998); Fonseca et al., “Flavivirus Type-Specific Antigens Produced from Fusions of a Portion of the E Protein Gene with the Escherichia Coli trpE Gene,” Am. J. Trop. Med. Hyg. 44(5):500-8 (1991); Megret et al., “Use of Recombinant Fusion Proteins and Monoclonal Antibodies to Define Linear and Discontinuous Antigenic Sites on the Dengue Virus Envelope Glycoprotein,” Virology 187(2):480-91 (1992), which are hereby incorporated by reference in their entirety). Based on results with DENV2 dIII protein it is expected that DENV dIII of all serotypes generally stimulate potent homotypic neutralizing antibodies that exhibit only trivial or no neutralizing activity against other DENV serotypes although this has not yet been formally determined. Therefore, the most potent neutralizing antibodies generated by the vaccine are predicted to be directed to the DENV dIII lateral ridge where DENV serotype specific epitopes are concentrated (Sukupolvi-Petty et al., “Type- and Subcomplex-Specific Neutralizing Antibodies Against Domain III of Dengue Virus Type 2 Envelope Protein Recognize Adjacent Epitopes,” J. Virol. 81(23):12816-26 (2007), which is hereby incorporated by reference in its entirety). However, the data also reveals cross-reactive antibodies that may exhibit little or no neutralizing activity. For example, DENV2 dIII generated antibodies that bound DENV1 (FIG. 12A) but did not neutralize it (FIG. 10D). Such antibodies might be directed to the DENV dIII AB loop region, which harbors DENV cross-reactive determinants that subserve marginal neutralizing activity (Sukupolvi-Petty et al., “Type- and Subcomplex-Specific Neutralizing Antibodies Against Domain III of Dengue Virus Type 2 Envelope Protein Recognize Adjacent Epitopes,” J. Virol. 81(23):12816-26 (2007), which is hereby incorporated by reference in its entirety).

The second main point that derived from the data is that the DENV dIII-specific neutralizing antibody response in mice appears somewhat divergent among different DENV serotypes, with DENV4 dIII uniformly stimulating the lowest titers. This was the case both when DENV4 dIII was delivered alone or in an equal-dose tetravalent formulation. Such DENV4 dIII immunogenic inferiority has also been observed by others (Lazo et al., “Dengue-4 Envelope Domain III Fused Twice Within the Meningococcal P64k Protein Carrier Induces Partial Protection in Mice,” Biotechnol. Appl. Biochem. 52(Pt 4):265-71 (2009), which is hereby incorporated by reference in its entirety), and remains unexplained. Differences in immunogenicity tied to the particular virus strains chosen might be an added contributing factor. Nevertheless, the balance among serotype specific neutralizing antibodies appeared to be modified by adjustments in the relative amount of each DENV dIII protein in the tetravalent formulations (e.g., DENV4 neutralization potency was significantly increased coincident with increasing the DENV4 dIII dose).

While increased representation by potent serotype-specific neutralizing antibodies against the DENV dIII lateral ridge may account for most of the improved neutralizing activity, it is plausible that some cross-reacting antibodies generated by the tetravalent preparation act synergistically to promote DENV4 neutralization. It is speculated that such synergy would have been further amplified were DENV4 dIII sequence homology greater. While the protection tests have not yet been conducted, the neutralization titers achieved by the tetravalent DENV dIII vaccine formulations should reasonably be expected to confer protection.

The third key finding of the preceding Examples is that DENV dIII vaccine immune sera promote ADE, albeit to a much lesser extent than attenuated live vaccines. In K562 cells, enhancement was relatively modest and similar to that mediated by antibodies from live DENV-immunized mice. In U937 cells, however, ADE mediated by live virus-stimulated antibodies was strikingly greater than that with DENV dIII vaccine antibodies. Concordantly, ADE mediated by an IgG2a DENV2 dIII specific mAb (1F1) was strikingly greater in U937 than in K562 cells. These disparities are not yet explained, but it is tempting to speculate that in U937 cells IgG2a antibodies subserve ADE more efficiently than do IgG1 antibodies which predominate in the DENV dIII vaccine sera.

Collectively the data confirms the feasibility of an effective DENV dIII subunit vaccine. Shaping the DENV dIII neutralizing antibody response to favor a more balanced DENV serotype specific repertoire is desirable and can be further enhanced by formulation with other adjuvants proven suitable for human use (Guy B., “The Perfect Mix: Recent Progress in Adjuvant Research,” Nat. Rev. Microbiol. 5(7):505-17 (2007), which is hereby incorporated by reference in its entirety). Additionally, since DENV dIII serotype-specific neutralizing antibody responses may differ, at least among DENV3 strains (Wahala et al., “Natural Strain Variation and Antibody Neutralization of Dengue Serotype 3 Viruses,” PLoS. Pathog. 6(3):e1000821 (2010); Brien et al., “Genotype-Specific Neutralization and Protection by Antibodies Against Dengue Virus Type 3,” J. Virol. 84(20):10630-43 (2010), both of which are hereby incorporated by reference in their entirety), more complex tetravalent formulations may be required to elicit comprehensively protective antibodies.

### Example 7

### Immune Responses Elicited by a Tetravalent Dengue Vaccine in Non-Human Primate

The preceding Examples demonstrated that immunization of mice with a tetravalent Dengue dIII vaccine elicited robust neutralizing antibody responses to all four dengue serotypes. In this follow-up study, it will be examined how immunogenic the same vaccine antigens are when formulated with aluminum adjuvant in macaques (e.g., Rhesus or cynomolgus). The primary objective of this study is to determine neutralization antibody responses elicited in vaccinated macaques. The secondary exploratory objectives include, but are not limited to, the assessment of cytokine response patterns in the vaccinated and control animals, as well as quantification of dengue-specific antibody-producing B cell numbers.

Each macaque will be injected IM with 500 μl of vaccine or control formulations (see Table 2 below). A total of three injections will be given at days 0, 14 and 28. Peripheral blood samples (10-15 ml per bleed, maximal amounts permissible within the safety limit) will be collected on heparinized tubes at days −7, 7, 21 and 42. The experiment will be terminated at day 42 and the animals will be transferred to other use at the discretion of the animal facility authority.

TABLE 2
Reagent Needed Per Sample

| | | Reagent amounts per monkey (lot 1, μl) | | | | | | |
|---|---|---|---|---|---|---|---|---|
| | Vaccine | Al- | | | | | | Total for 6 |
| Groups | (μg) | hydrogel | DENV1 | DENV2 | DENV3 | DENV4 | PBS | animals(μg) |
| 1 | 0 | 100 | 0 | 0 | 0 | 0 | 400 | |
| (n = 2) | | | | | | | | |
| 2 | 50 each | 100 | 56.2 | 80.6 | 104 | 73.5 | 85.2 | 300 |
| (n = 4) | | | | | | | | |

| | | | | | |
|---|---|---|---|---|---|
| Total each injection (n = 6) | 337 | 484 | 624 | 441 | 900 |
| For 3 injections | 1012 | 1451 | 1872 | 1323 | |

Blood samples will be processed to plasma and peripheral blood mononuclear cell fractions and stored at −20° C. and −150° C. for plasma and cells, respectively. The plasma will be used to perform a modified PRNT assay as described (Rodrigo et al., “Differential Enhancement of Dengue Virus Immune Complex Infectivity Mediated by Signaling-Competent and Signaling-Incompetent Human Fcγ RIA (CD64) or FcγRIIA (CD32),” J. Virol. 80(20):10128-38 (2006), which is hereby incorporated by reference in its entirety), and PRNT₅₀ will be determined and compared between control and vaccine groups, and among sampling time-points. Part of the blood samples will also be used to profile cytokine secretion patterns as describe (Kou et al., “Human Antibodies Against Dengue Enhance Dengue Viral Infectivity Without Suppressing Type I Interferon Secretion in Primary Human Monocytes,” Virology 410(1):240-7 (2011), which is hereby incorporated by reference in its entirety). Cells will be used to perform exploratory assay, including but not limited, to the measurement of dengue DIII-specific antibody-producing B cell numbers (see Table 2 above).

Reagents include sterile 2% Alhydrogel (Accurate Chemical & Scientific Corp.), DENV domain III proteins (prepared as described in Example 1), and sterile PBS (DPBS, GIBCO) (see Table 3 below).

TABLE 3
DENV-1, 2, 3, and 4 Concentrations

| | Lot 1 | | Lot 2 | |
|---|---|---|---|---|
| | Concentration | Aliquot size | Concentration | Aliquot size |
| | (μg/ml) | (μl) | (μg/ml) | (μl) |
| DENV-1 | 890 | 1,000 | 2890 | 400 |
| DENV-2 | 620 | 1,000 | 2880 | 400 |
| DENV-3 | 480 | 1,000 | 1180 | 1600 |
| DENV-4 | 680 | 1,000 | 1230 | 1600 |

### Example 8

### Monovalent Yellow Fever Virus 17D dIIII Immunization Elicits Homologous Virus Neutralizing Antibodies

The procedures described in Example I were used to generate purified, recombinant YFV17D dIII polypeptide for use in a monovalent vaccine against YFD. The YFV17D dIII nucleotide sequence is shown below as SEQ ID NO: 59 below.

| | | |
|---|---|---|
| 1 | TCCTACAAAATATGCACTGACAAAATGTTTTTTGTCAAGAACCCAACTGA | 50 |
| 51 | CACTGGCCATGGCACTGTTGTGATGCAGGTGAAAGTGTCAAAAGGAGCCC | 100 |
| 101 | CCTGCAGGATTCCAGTGATAGTAGCTGATGATCTTACAGCGGCAATCAAT | 150 |
| 151 | AAAGGCATTTTGGTTACAGTTAACTCCATCGCCTCAACCAATGATGATGA | 200 |
| 201 | AGTGCTGATTGAGGTGAACCCACCTTTTGGAGACAGCTACATTATCGTTG | 250 |
| 251 | GGAGAGGAGATTCACGTCTCACTTACCAGTGGCACAAAGAGGGATCC | 297 |

The encoded dIII polypeptide has the amino acid sequence of SEQ ID NO: 40 as follows:

| | | |
|---|---|---|
| 1 | SYKICTDKMFFVKNPTDTGHGTVVMQVKVSKGAPCRIPVIVADDLTAAIN | 50 |
| 51 | KGILVTVNSIASTNDDEVLIEVNPPFGDSYIIVGRGDSRLTYQWHKEGS | 100 |

Briefly, Trichoplusia ni insect cells (High Five™ cells, Invitrogen, Carlsbad, Calif.) were propagated in 300-mL shake cultures (125 rpm, 27° C.) in Express Five serum-free medium (Invitrogen) and were infected at a multiplicity of infection (MOI)=3. Cell cultures were incubated with shaking for 72 hours at 27° C. Supernatant containing secreted recombinant YFV17D dIII protein was clarified by centrifugation (800×g) and incubated with Talon metal affinity resin (Talon Metal Affinity Purification, BD Biosciences, Palo Alto, Calif.) for metal affinity chromatography. Protein was eluted from beads using 10 mM imidazole and dialyzed against PBS. Protein concentration was determined by bicinchoninic acid assay (Pierce, Rockford, Ill.). Recombinant protein (200 ng) was resolved by 15% SDS-PAGE and visualized with Coomassie brilliant blue (Sigma, St. Louis, Mo.). Protein were transferred to nitrocellulose membranes for immunoblot.

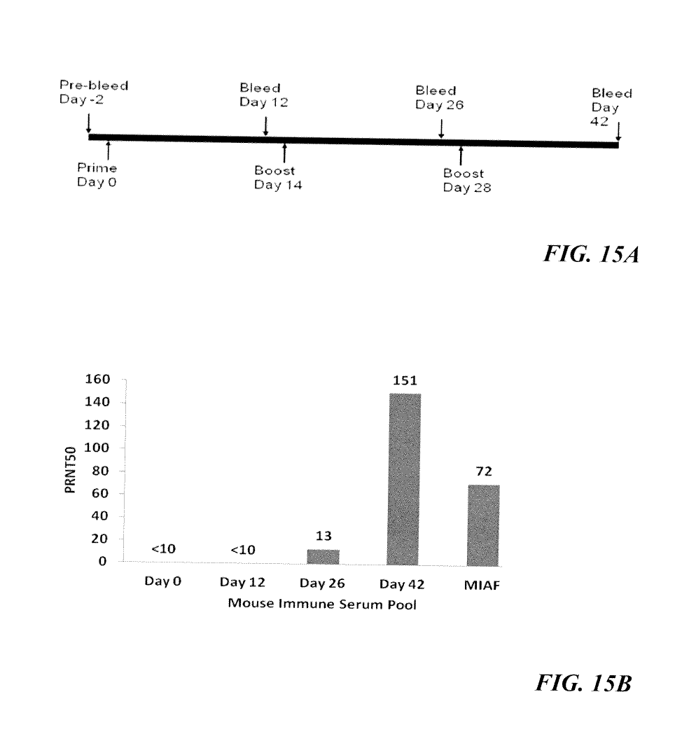

50 μg of recombinant YFV17D dIII protein was emulsified in complete Freund's adjuvant (CFA, Sigma, St. Louis, Mo.) for priming (day 0), and in incomplete Freund's adjuvant (IFA) for booster immunizations (days 14 and 28). See FIG. 15A. Protein doses were delivered by hind leg intramuscular (i.m.) injection. Blood was collected on day −2, 12, and 26 by retro-orbital bleed, and by terminal cardiac puncture on day 42.

Antibody-mediated YFV17D neutralization in Vero cells was determined by a previously described microneutralization plaque assay in Vero cells using an anti-YFV17D NS1 monoclonal antibody to immunostain YFV17D plaques (Shanaka et al., “An Automated Dengue Virus Microneutralization Plaque Assay Performed in Human Fc{Gamma} Receptor-Expressing CV-1 Cells,” Am. J. Trop. Med. Hyg. 80(1):61-5 (2009), which is hereby incorporated by reference in its entirety). Percent plaque reduction and PRNT₅₀ titers were calculated by probit analysis using GraphPad Prism software v5.0 as described above.

Neutralization assays with pooled sera collected on days −2, 10, 26, and 42, demonstrated potent homotypic neutralizing activity against YF-dIII (FIG. 15B). Pre-immune sera and sera collected on day 10 had undetectable PRNT₅₀ titers (>10) whereas sera collected on day 26 following the initial boost (PRNT₅₀=13) and following the second booster immunization on day 42 (PRNT₅₀=151) had increasingly higher neutralization titers. As a comparator, YF17D mouse immune ascitic fluid (MIAF) harvested from YFV17D virion vaccinated mice exhibited similar, albeit lower, neutralizing antibodies (PRNT₅₀=72).

Importantly, such antibody levels generated by the dIII subunit YFV17D vaccine is comparable to that observed in human subjects immunized with an experimental inactivated whole virion YFV17D vaccine (day 42; PRNT₅₀= 1/113) (Monath et al., “An Inactivated Cell-culture Vaccine Against Yellow Fever,” New Engl. J. Med. 364(14):1326-33 (2011), which is hereby incorporated by reference in its entirety). Since the minimal yellow fever protective neutralizing antibody level is widely accepted to be PRNT₅₀≧ 1/10 or 1/20, the YFV17D dIII antibody response is predicted to be protective.

### Example 9

### Pentavalent Dengue and Yellow Fever Virus Vaccine

Since mosquito-borne dengue and yellow fever viruses co-circulate in regions of Africa and South America, a pentavalent subunit vaccine comprised of DENV1-4 and YFV17D dIII polypeptides will be formulated using 50 gi/dose YFV17D dIII polypeptide added to the formulation tetravalent DENV1-4 dIII formulation of Example 7. Based on the data presented herein, it is expected that the pentavalent vaccine formulation will confer broad protection against these viruses.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A tetravalent Dengue virus vaccine comprising:
a Dengue domain III polypeptide for each of DEN1 to DEN4, wherein the vaccine induces a neutralizing antibody response against each of DEN1 to DEN4 that exceeds PRNT₅₀ value of 200.

2. The tetravalent Dengue virus vaccine according to claim 1, wherein the Dengue domain III polypeptide for one or more of DEN1 to DEN4 is an isolated polypeptide.

3. The tetravalent Dengue virus vaccine according to claim 1, wherein the Dengue domain III polypeptide for one or more of DEN1 to DEN4 is a fusion protein.

4. The tetravalent Dengue virus vaccine according to claim 3, wherein the fusion protein comprises an adjuvant polypeptide.

5. The tetravalent Dengue virus vaccine according to claim 4, wherein the adjuvant polypeptide is selected from the group consisting of flagellin, human papillomavirus L1 or L2 protein, herpes simplex glycoprotein D (gD), complement C4 binding protein, TLR-4 ligand, and IL-1β.

6. The tetravalent Dengue virus vaccine according to claim 3, wherein the fusion protein comprises two or more Dengue domain III polypeptides.

7-8. (canceled)

9. The tetravalent Dengue virus vaccine according to claim 1, wherein:
the Dengue domain III polypeptide for DEN1 comprises the amino acid sequence according to SEQ ID NO: 1 or an amino acid sequence sharing at least 85% identity to SEQ ID NO: 1;
the Dengue domain III polypeptide for DEN2 comprises the amino acid sequence according to SEQ ID NO: 7 or an amino acid sequence sharing at least 85% identity to SEQ ID NO: 7;
the Dengue domain III polypeptide for DEN3 comprises the amino acid sequence according to SEQ ID NO: 15 or an amino acid sequence sharing at least 85% identity to SEQ ID NO: 15; and/or
the Dengue domain III polypeptide for DEN4 comprises the amino acid sequence according to SEQ ID NO: 22 or an amino acid sequence sharing at least 85% identity to SEQ ID NO: 22.

10-12. (canceled)

13. The tetravalent Dengue virus vaccine according to claim 1, wherein one or more of the Dengue domain III polypeptides for DEN1 to DEN4 is conjugated to an immunogenic carrier molecule.

14. (canceled)

15. The tetravalent Dengue virus vaccine according to claim 13, wherein the carrier molecule is selected from the group of bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein.

16. The tetravalent Dengue virus vaccine according to claim 1 further comprising an adjuvant.

17. The tetravalent Dengue virus vaccine according to claim 16, wherein the adjuvant is selected from the group consisting of flagellin, Freund's complete or incomplete adjuvant, aluminum, aluminum hydroxide, lysolecithin, pluronic polyols, polyanions, peptides, oil emulsion, dinitrophenol, iscomatrix, ASO4, papillomavirus VLPs or capsomeres, and liposome polycation DNA particles.

18. The tetravalent Dengue virus vaccine according to claim 1 further comprising a pharmaceutically acceptable carrier.

19. The tetravalent Dengue virus vaccine according to claim 18, wherein the carrier is selected from the group consisting of solutions, suspensions, emulsions, excipients, powders, and stabilizers.

20. The tetravalent Dengue virus vaccine according to claim 1 further comprising a delivery vehicle.

21. The tetravalent Dengue virus vaccine according to claim 20, wherein the delivery vehicle is selected from the group consisting of biodegradable microspheres, microparticles, nanoparticles, liposomes, collagen minipellets, and cochleates.

22. The tetravalent Dengue virus vaccine according to claim 20, wherein the vaccine is in the form of a single-unit oral, nasal, injectable, or aerosolized dosage.

23. (canceled)

24. A multivalent vaccine comprising:
an effective amount of a Dengue domain III polypeptide for each of DEN1 to DEN4,
an effective amount of a Yellow Fever domain III polypeptide, and
a pharmaceutically acceptable carrier.

25. The multivalent vaccine according to claim 24, wherein the vaccine induces a neutralizing antibody response against each of DEN1 to DEN4 and YFV that exceeds a PRNT₅₀ value of 150.

26. A method of inducing a neutralizing immune response against Dengue virus 1-4 in a subject comprising:
administering to the subject a tetravalent Dengue virus vaccine according to claim 1 in an amount effective to induce a neutralizing immune response against each of DEN1 to DEN4 that exceeds PRNT₅₀ of 200.

27. The method according to claim 26, wherein said administering is carried out orally, by inhalation, by intranasal instillation, topically, transdermally, parenterally, subcutaneously, intravenous injection, intra-arterial injection, intramuscular injection, intraplurally, intraperitoneally, or by application to mucous membrane.

28-31. (canceled)

32. A method of making a vaccine comprising:
introducing into a host cell a transgene that encodes a recombinant protein comprising a secretion signal in-frame with a Flavivirus domain III (dIII) polypeptide;
growing the host cell under conditions effective to express the recombinant protein in soluble form;
recovering the recombinant protein in the absence of one or more refolding steps;
cleaving the secretion signal from the recovered protein to obtain a dIII polypeptide; and
combining, with a pharmaceutically acceptable vehicle, dIII polypeptide specific for multiple Flaviviruses in amounts effective to induce a neutralizing immune response against each of the Flaviviruses that exceeds PRNT₅₀ of 150.

33. (canceled)

34. The method according to claim 32 further comprising purifying the dIII polypeptide prior to said combining.

35. The method according to claim 34, wherein the dIII polypeptide comprises a purification tag.

36. The method according to claim 32 wherein the host cell is an insect cell and the transgene is present in a recombinant baculovirus vector.

37. (canceled)

38. The method according to claim 32 further comprising:
introducing an adjuvant into the pharmaceutically acceptable vehicle.

39. The method according to claim 32, wherein the Flaviviruses comprise two or more of Dengue virus-1, Dengue virus-2, Dengue virus-3, Dengue virus-4, Yellow Fever virus, West Nile virus, and Japanese Encephalitis virus.

40. The method according to claim 32, wherein the Flaviviruses comprise each of Dengue virus-1, Dengue virus-2, Dengue virus-3, and Dengue virus-4.

41. The method according to claim 32, wherein the Flaviviruses comprise each of Dengue virus-1, Dengue virus-2, Dengue virus-3, Dengue virus-4, and Yellow Fever virus.

42-50. (canceled)
